(19) United States
(12) Patent Application Publication
(10) Pub. No.: US 2024/0335437 A1
(43) Pub. Date: Oct. 10, 2024

(54) SUSTAINED RELEASE STABLE EMULSION PHARMACEUTICAL FORMULATIONS

(71) Applicant: InSitu Biologics, Inc., Oakdale, MN (US)

(72) Inventors: William J. Taylor, Woodbury, MN (US); Kelsey Pflepsen, Minneapolis, MN (US)

(21) Appl. No.: 18/631,935

(22) Filed: Apr. 10, 2024

Related U.S. Application Data
(60) Provisional application No. 63/458,349, filed on Apr. 10, 2023.

(51) Int. Cl. A61K 31/445; A61K 9/107; A61K 47/10; A61K 47/14; A61K 47/24; A61K 47/26

(52) U.S. Cl. CPC A61K 31/445; A61K 9/107; A61K 47/10; A61K 47/14; A61K 47/24; A61K 47/26

(57) ABSTRACT

Disclosed herein is composition for treating pain in a subject in need thereof that comprises an emulsion comprising: an aqueous carrier; and liquid lipid phase dispersed into droplets within the aqueous carrier, and a first anesthetic agent within the lipid phase. In certain embodiments, the first anesthetic agent comprises a first plurality of anesthetic agent crystals. In certain alternative embodiments, the first anesthetic agent is dissolved with the lipid phase. In certain implementations, the composition further comprises a second plurality of anesthetic agent crystals within the aqueous carrier, but not the lipid phase and wherein the second plurality of anesthetic agent crystals dissolves and elutes from the emulsion at a faster rate than the first plurality anesthetic agent crystals. In certain implementations, the composition further comprises one or more additional anesthetic agents, different from the first anesthetic agent.

### CROSS-REFERENCE TO RELATED APPLICATION(S)

This application claims priority to U.S. Provisional Application No. 63/458,316, filed Apr. 10, 2023, and entitled “SUSTAINED RELEASE STABLE EMULSION PHARMACEUTICAL FORMULATIONS,” which is hereby incorporated by reference in its entirety under 35 U.S.C. § 119 (e).

### BACKGROUND

Liposomal bupivacaine has been used in peripheral nerve blocks to prolong duration of action of the local anesthetic and to reduce post-operative pain and perioperative opioid use. In practice, that preparation often falls short of eliminating opiate use for surgical patients in many settings. The pharmacokinetic profile of the delivery system and migration of the local anesthetic in tissue jointly contribute to limited efficacy and duration of action at target sites.

Acute post-op pain is estimated to need at least 5-7 days of pain relief coverage. To that end, advancements in perioperative medicine endeavor to eliminate opioids from a post-operative treatment regimen but have thus far fallen. Numerous opioid-limiting techniques such as intrathecal morphine, medicinal adjuncts, and regional anesthetics of standard and extended-release preparations demonstrate limited effectiveness to 24-48 hours of effective analgesia-often requiring narcotics to supplement waning analgesia.

There is a need in the art for compositions and methods that are effective in providing sustained post-operative pain relief without the use of opiate based medications.

### BRIEF SUMMARY

While multiple embodiments are disclosed, still other embodiments of the disclosure will become apparent to those skilled in the art from the following detailed description, which shows and describes illustrative embodiments of the disclosed apparatus, systems, and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 shows porcine ropivacaine blood plasma concentration (ng/ml) after sciatic peripheral nerve block procedure with the instantly disclosed stable emulsion (6.29 mg/Kg formulation) compared to Naropin 0.5% positive control, according to certain embodiments.

FIG. 2 shows porcine ropivacaine blood plasma concentration (ng/mL) over time following injection of instantly disclosed stable emulsion (20.44 mg/Kg formulation) or Naropin 0.5% positive control as peripheral nerve block, according to certain embodiments.

FIG. 3A shows a microscope image of Sample 1 at 10× magnification, according to certain implementations.

FIG. 3B shows a microscope image of Sample 1 at 20× magnification, according to certain implementations.

FIG. 4A shows a microscope image of Sample 2 at 10× magnification, according to certain implementations.

FIG. 4B shows a microscope image of Sample 2 at 20× magnification, according to certain implementations.

FIG. 5A shows a microscope image of Sample 3 at 10× magnification, according to certain implementations.

FIG. 5B shows a microscope image of Sample 3 at 20× magnification, according to certain implementations.

FIG. 6A shows a microscope image of Sample 4 at 10× magnification, according to certain implementations.

FIG. 6B shows a microscope image of Sample 4 at 20× magnification, according to certain implementations.

FIG. 7A shows a microscope image of Sample 5 at 10× magnification, according to certain implementations.

FIG. 7B shows a microscope image of Sample 5 at 20× magnification, according to certain implementations.

FIG. 8A shows a microscope image of Sample 6 at 10× magnification, according to certain implementations.

FIG. 8B shows a microscope image of Sample 6 at 20× magnification, according to certain implementations.

FIG. 9A shows a microscope image of Sample 7 at 10× magnification, according to certain implementations.

FIG. 9B shows a microscope image of Sample 7 at 20× magnification, according to certain implementations.

FIG. 10A shows a microscope image of Sample 8 at 10× magnification, according to certain implementations.

FIG. 10B shows a microscope image of Sample 8 at 20× magnification, according to certain implementations.

FIG. 11A shows a microscope image of Sample 9 at 10× magnification, according to certain implementations.

FIG. 11B shows a microscope image of Sample 9 at 20× magnification, according to certain implementations.

FIG. 12A shows a microscope image of Sample 10 at 10× magnification, according to certain implementations.

FIG. 12B shows a microscope image of Sample 10 at 20× magnification, according to certain implementations.

FIG. 13A shows a microscope image of Sample 11 at 10× magnification, according to certain implementations.

FIG. 13B shows a microscope image of Sample 11 at 20× magnification, according to certain implementations.

FIG. 14A shows a microscope image of Sample 12 at 10× magnification, according to certain implementations.

FIG. 14B shows a microscope image of Sample 12 at 20× magnification, according to certain implementations.

FIG. 15A shows a microscope image of Sample 13 at 10× magnification, according to certain implementations.

FIG. 15B shows a microscope image of Sample 13 at 20× magnification, according to certain implementations.

FIG. 16A shows a microscope image of Sample 14 at 10× magnification, according to certain implementations.

FIG. 16B shows a microscope image of Sample 14 at 20× magnification, according to certain implementations.

FIG. 17A shows a microscope image of Sample 15 at 10× magnification, according to certain implementations.

FIG. 17B shows a microscope image of Sample 15 at 20× magnification, according to certain implementations.

FIG. 18A shows a microscope image of Sample 16 at 10× magnification, according to certain implementations.

FIG. 18B shows a microscope image of Sample 16 at 20× magnification, according to certain implementations.

FIG. 19A shows a microscope image of Sample 17 at 10× magnification, according to certain implementations.

FIG. 19B shows a microscope image of Sample 17 at 20× magnification, according to certain implementations.

FIG. 20A shows a microscope image of Sample 18 at 10× magnification, according to certain implementations.

FIG. 20B shows a microscope image of Sample 18 at 20× magnification, according to certain implementations.

FIG. 21A shows a microscope image of Sample 19 at 10× magnification, according to certain implementations.

FIG. 21B shows a microscope image of Sample 19 at 20× magnification, according to certain implementations.

FIG. 22A shows a microscope image of Sample 20 at 10× magnification, according to certain implementations.

FIG. 22B shows a microscope image of Sample 20 at 20× magnification, according to certain implementations.

FIG. 23A shows a microscope image of Sample 21 at 10× magnification, according to certain implementations.

FIG. 23B shows a microscope image of Sample 21 at 20× magnification, according to certain implementations.

FIG. 24A shows a microscope image of Sample 22 at 10× magnification, according to certain implementations.

FIG. 24B shows a microscope image of Sample 22 at 20× magnification, according to certain implementations.

FIG. 25A shows a microscope image of Sample 23 at 10× magnification, according to certain implementations.

FIG. 25B shows a microscope image of Sample 23 at 20× magnification, according to certain implementations.

FIG. 26 shows a microscope image of Sample 24 at 40× magnification, according to certain implementations.

FIG. 27 shows a microscope image of Sample 25 at 40× magnification, according to certain implementations.

### DETAILED DESCRIPTION

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present disclosure, example methods and materials are now described.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Perkin Elmer Corporation, U.S.A.).

As used in the specification, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Unless otherwise indicated, references in the specification to parts by weight of a particular element or component in a composition denotes the weight relationship between the element or component and any other elements or components in the composition or article for which a part by weight is expressed, unless expressly described otherwise. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

The term “anesthetic agent” or “local anesthetic agent” (used unteachably herein) refers to an agent that causes loss of sensation in a human or other mammal with or without the loss of consciousness. More particularly, the term “local anesthetic” refers to an anesthetic agent that induces local anesthesia by reversibly inhibiting peripheral nerve excitation and/or conduction. Local anesthetics suitable for use in the present invention include, but are not limited to, ester-based anesthetics, amide-based anesthetics, ester analogs of amide-based anesthetics, and ester analogs of other anesthetics. Ester-based anesthetics include, but are not limited to, cocaine, procaine, 2-chloroprocaine, tetracaine, benzocaine, amethocaine, chlorocaine, butamben, dibucaine, and the like. Amide-based anesthetics include, but are not limited to, lidocaine, prilocaine, mepivacaine, ropivacaine, etidocaine, levobupivacaine, bupivacaine, and the like. Other anesthetics suitable for use in the present invention include, but are not limited to, ester analogs of aconitine, dyclonine, ketamine, pramoxine, safrole, and salicyl alcohol. Such ester analogs can contain an ester group anywhere within the structure.

As used herein, the term “subject” refers to the target of administration, e.g. a subject. Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the terms “treat,” and “prevent” as well as words stemming therefrom, do not necessarily imply 100% or complete treatment or prevention. Rather, there are varying degrees of treatment or prevention of which one of ordinary skill in the art recognizes as having a potential benefit or therapeutic effect. In this respect, the methods of the present invention can provide any amount of any level of treatment or prevention of a disease or medical condition in a mammal. Furthermore, the treatment or prevention provided by the method can include treatment or prevention of one or more conditions or symptoms of the disease or medical condition. For example, with regard to methods of treating pain, the method in some embodiments, achieves a diminution in or elimination of pain in a subject. Also, for purposes herein, “prevention” can encompass delaying the onset of the disease, or a symptom or condition thereof. The term “treating” includes prophylaxis of the specific disorder or condition, or alleviation of the symptoms associated with a specific disorder or condition and/or preventing or eliminating said symptoms. For example, as used herein the term “post-operative pain” refers in general to producing a diminution or alleviation of pain associated with recovering from a surgical procedure.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of particles would either completely lack particles, or so nearly completely lack particles that the effect would be the same as if it completely lacked particles. In other words, a composition that is “substantially free of an ingredient or element may still actually contain such item as long as there is no measurable effect thereof.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

Effective dosages may be estimated initially from in vitro assays. For example, an initial dosage for use in animals may be formulated to achieve a circulating blood or serum concentration of active compound that is at or above an IC50 of the particular compound as measured in an in vitro assay. Calculating dosages to achieve such circulating blood or serum concentrations, taking into account the bioavailability of the particular active agent, is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pergamagon Press, which is hereby incorporated by reference in its entirety, and the references cited therein.

Pharmaceutically active small molecules and biological compounds are eluted from custom formulated multi-phase stable emulsions to produce a biological response in the organism such as a peripheral nerve block, or if infiltrated at a wound site, prolonged pain relief for multiple days. Depending on active pharmaceutical ingredient contained within the drug reservoir, other useful treatments, and clinical applications other than pain relief may be provided for multiple days or even longer. In general, a stable emulsion containing solid crystal pharmaceutical agents and or solid phase drug reservoir are contained within a continuous carrier phase that forms a liquid emulsion that allows delivery via syringe and hypodermic needle, through a catheter or through an applicator. Alternative forms may have liquid, solid, or semisolid drug reservoirs or mixtures of multiple drug reservoir forms within the emulsion.

In certain embodiments, the instantly disclosed drug product is a stable 3 phase emulsion formulation that provides excellent sustained release performance for small molecules like ropivacaine and bupivacaine but with the advantages of not having a residual active pharmaceutical ingredient (API) or drug product excipient remaining in the body after 14 days. As used herein, API generally refers to an anesthetic agent, although in certain embodiments, non-anesthetic agent APIs are possible. It is advantageous to use the body's tissues to absorb and remove the drug product to prevent irritation or compression of the nerve or surrounding tissues during movement of neighboring muscles. According to certain embodiments, the instantly disclosed emulsion is primarily designed to act as a peripheral nerve block (PNB) in which it can produce a full nerve block within the first hour post administration and maintain a pain block for at least 3 days (72 hrs). The ability of a PNB to produce a pain block for at least three days allows the patient to avoid opioids and transition to non-opioid medications after 3 days.

No other drug formulations have yet been capable of producing both a strong nerve block and prolonged pain relief for at least 72 hours. They may be able to treat pain locally at the wound or in some examples require additional/supplemental anesthetic to produce a strong block. The stable emulsion formulation drug product described herein produces a fast onset robust nerve block followed by at least 3 days pain anesthetic. It is absorbed by the body within 14 days and does not compress or cause inflammation at the injection site.

The unique composition of the drug product allows it to store and retain the API, ropivacaine in these examples, and limit its elution rates. Solid particles within emulsions usually allow for coalescence of a lipid phase and aqueous phases and destabilizes emulsions. In some embodiments, the solids particles can be drug crystals. In other embodiments, the solid particles can be solid microparticles. In various other embodiments, the solid particles can be various other configurations that would be understood as functionally compatible with the formulation by those skilled in the art. In the described formulations, the solid particles do not destabilize the emulsion and the emulsions retain the solid particles dispersed within and throughout the two liquid phases in a homogenous liquid.

Typically, emulsions are not able to deliver a sufficient dose of API to last several days as they cannot carry solid particles and are thus limited in the amount of drug carried in a single dose. Stable emulsions typically do not contain solid particles that may initiate coalescence of the lipid and aqueous phases. The instantly disclosed formulation is unique in that it can hold solid API particles in the emulsion up to 28% by weight relative to the lipid components or 12% relative to the entire drug product in a stable emulsion, as defined below. In some formulations the emulsion can carry up to 30% API, or 15% relative to entire drug product volume, but they become thick and must be delivered through larger gauge needles and applicators. Similarly, many lipid components such as triglycerides have low API solubility at body and ambient temperatures preventing them from dissolving pharmaceutically effective amounts of API. Due to its unique manufacturing process, the instantly disclosed formulation creates a stable emulsion with solid API crystals.

In certain embodiments, API crystals are suspended in the emulsions with a small amount of dissolved API present in both the lipid and aqueous phases. One of the main barriers to elution, which creates the extended-release profile, is the physical transformation of the solid API crystal phase into the liquid phases of the reservoir lipid droplets or the continuous aqueous phase. A lipophilic API molecule, such as ropivacaine, may have a low solubility in both lipid and aqueous phases. The dissolved API present in each phase is eluted into the surrounding tissue. Immediately following injection, this transformation of API crystals will occur at a faster rate due to the chemical gradient between the emulsion (high ropivacaine) and surrounding tissue (low ropivacaine). Dissolved API will readily elute into surrounding tissue; thereby decreasing the dissolved API present in the lipid and aqueous phases. This, in turn, creates a chemical gradient between the crystals and the lipid and aqueous phases, and the solid API crystals will then dissolve into each phase at a similar rate to the elution into surrounding tissue from each phase. In addition to the above-mentioned processes, transfer of dissolved API at the liquid/liquid interface between one low solubility liquid phase into another low solubility liquid may also be occurring. Once the chemical gradient between the emulsion and surrounding tissue has decreased due to the elution of ropivacaine, the above-mentioned processes will reach a slow steady state until all API crystals have been dissolved, at which point, the API elution rate will be below the pharmaceutically effective dose locally. For typical formulations with ropivacaine as the API, ropivacaine levels in plasma remain at detectable levels for up to about 7 days and necropsy results show the drug product has been cleared or mostly cleared from the injection space within about 14 days.

According to certain implementations, the disclosed stable emulsion comprises an aqueous carrier; and lipid phase dispersed into droplets within the aqueous carrier, and a first plurality anesthetic agent crystals within the lipid phase. In certain implementations, a second plurality of API crystals within the aqueous carrier, but not the lipid phase and wherein the second plurality of API crystals dissolves and elutes from the stable emulsion at a faster rate than the first plurality API crystals.

An emulsion can be considered stable if the immiscible phases of the remain unseparated for a commercially useful period. A commercially useful period is one that allows for sufficient time for all relevant testing, shipping, storage, and use to take place. A commercially useful period, in some embodiments, can be between about 1 week to about 5 years. In further embodiments, a commercially useful period can be between about 1 month to about 2 years. In a preferred embodiment, a commercially useful period can be between about 6 months to about 12 months.

An emulsion can also be reversible. A reversible emulsion is one, whereupon the once-emulsified immiscible phases have separated, they can be returned to a stable, unseparated state with mixing gentler than required to initially combine the immiscible phases. In some instances, this mixing needed to recombine the separated phases can be accomplished by shaking the container, although more vigorous mixing may be needed.

In some embodiments, the presence of anesthetic agent crystals can cause an increase in formulation viscosity. In some embodiments, the increasing presence of anesthetic agent crystals can increase the viscosity of the formulation to about 150 cP to about 500 cP. In further embodiments, the increasing presence of anesthetic agent crystals can increase the viscosity of the formulation to about 220 cP.

### Lipid Phase

According to certain embodiments, the lipid phase is dispersed within the aqueous phase and contains anesthetic agent within the dispersed droplets. In certain embodiments, the anesthetic agent is present in the lipid phase in the form of anesthetic agent crystals. In further embodiments, the anesthetic agent is dissolved within the lipid phase. The lipid phase may comprise one or more triglycerides and is a liquid at room temperature.

### Aqueous Phase

According to certain embodiments, the aqueous phase can be dispersed as droplets within a lipid liquid continuous phase. The anesthetic agent may be present in the aqueous phase as a solute and also may be present in solid crystal phase. Multiple anesthetic agents may be present in the aqueous phase droplets. An additional lipophilic anesthetic agent may be present in the lipid continuous phase along with hydrophilic anesthetic agents in the aqueous droplets.

### Triglycerides

Triglycerides are an ester of glycerol and three fatty acids that can be all the same fatty acid or mixtures of different fatty acids. The fatty acids can be saturated (no double carbon bonds in the fatty acid chain) or unsaturated (one or more carbon double bonds in the fatty acid chain length) or mixtures of both saturated and unsaturated fatty acid chains. Triglycerides are the primary component in animal fats and plant derived oils.

Triglycerides have long been used as carriers for pharmaceutical ingredients. Castor oil, soy oil, and peanut oils have all been used as carriers for hydrophobic anesthetic agents. These formulations are used when a pharmaceutically effective dose can be dissolved within the carrier volume and the volume is small enough to not cause significant discomfort in the recipient.

Triglycerides have been used as a nutritional supplement intravenously or orally and are one of the main energy storage molecules in mammals. Stable emulsions are used in the pharmaceutical industry, but most do not contain solid anesthetic agent crystals in the formulations.

TABLE 1
Pure Triglycerides

| | Fatty Acid | | Fatty Acid |
|---|---|---|---|
| Common Name | Component | Structure | Saturation |
| Tripropionin | Propanoic acid | C12H20O6 | C3:0 |
| Tributyrin | Butyric acid | C15H26O6 | C4:0 |
| Trivalerin | Valeric acid | C18H32O6 | C5:0 |
| Tricaproin | Caproic acid | C15H26O6 | C6:0 |
| Triheptanoin | Heptanoic acid | C24H44O6 | C7:0 |
| Tricaprylin | Caprylic acid | C₂₇H₅₀O₆ | C8:0 |
| Tripelarigonin | Pelargonic acid | C30H56O6 | C9:0 |
| Tricaprin | Decanoic acid | C₃₃H₆₂O₆ | C10:0 |
| Triundcylin | Undecanoic acid | C36H68O6 | C11:0 |
| Trilaurin | Lauric acid | C39H74O6 | C12:0 |
| Tritridecanoin | Tridecanoic acid | C42H80O6 | C13:0 |
| Trimyristin | Myristic acid | C45H86O6 | C14:0 |
| Tripentadecanoin | Pentadecanoic acid | C48H92O6 | C15:0 |
| Tripalmitin | Palmitic acid | C51H98O6 | C16:0 |
| Trimargarin | Margaric acid | C54H104O6 | C17:0 |
| Tristearin | Stearic acid | C57H110O6 | C18:0 |
| Triolein | Oleic acid | C57H104O6 | C18:1, cis 9 |
| Trinonadecanoylglycerol | Nonadecanoic acid | C60H116O6 | C19:0 |
| Triarachidin | Arachidic acid | C63H122O6 | C20:0 |
| Triheneicosanoin | Heneicosylic acid | C66H128O6 | C21:0 |
| Trierucin | Erucic Acid | C69H128O6 | C22:cis13, 22:1ω9 |
| Tribehenin | Docosanoic acid | C69H134O6 | C22:0 |
| Tritricosanoin | Tricosanoic acid | C72H140O6 | C23:0 |
| Trilignocerin | Lignoceric acid | C75H146O6 | C24:0 |
| Tripentacosylin | Pentacosylic acid | C78H152O6 | C25:0 |
| Tricerotin | Cerotic acid | C81H158O6 | C26:0 |
| Tricarocerin | Carboceric acid | C84H164O6 | C27:0 |
| Trimontanin | Montanic acid | C87H170O6 | C28:0 |
| Trinonacosylin | Nonacosylic acid | C90H176O6 | C29:0 |
| Trimelissin | Melissic acid | C93H182O6 | C30:0 |
| Trihentriacontylin | Hentriacontylic acid | C96H188O6 | C31:0 |
| Trilacceroin | Lacceroic acid | C99H194O6 | C32:0 |
| Tripsyllin | Psyllic acid | C102H200O6 | C33:0 |
| Trigeddin | Geddic acid | C105H206O6 | C34:0 |
| Tricerplastin | Ceroplastic acid | C108H212O6 | C35:0 |
| Trihexatriacontylin | Hexatriacontylic acid | C111H218O6 | C36:0 |
| Triheptatriacontylin | Heptatriacontylic | C114H224O6 | C37:0 |
| Trioctatriacontylin | Octatriacontylic acid | C117H230O6 | C38:0 |
| Trinonatriacontlyin | Nonatriacontylic acid | C120H236O6 | C39:0 |
| Tritetracontylin | Tetracontylic acid | C122H242O6 | C40:0 |
| Triisopalmitin | Isopalmitic acid | C₅₁H₉₈O₆ | C16:0 |
| Triisostearin | Isostearic acid | C57H110O6 | C18:0 |
| Trilinolein | Linoleic acid | C57H98O6 | C18:2n-6 |
| Triheptylundecanoin | Heptylundecanoic | C57H110O6 | C18:0 |
| Tripalmitolein | Palmitoleic acid | C51H92O6 | C16:1-8 |
| Triricinolein | Ricinoleic acid | C57H104O9 | C18:1-9, 11-OH |

TABLE 2
Mixed Triglycerides

| Oil | Primary Fatty Acid Components | Source |
|---|---|---|
| Soybean | 51% linoleic acid, 7-10% α- | Soybean |
| | linolenic acid, 23% oleic acid, 10% | (Glycine max) |
| | palmitic acid and 4% stearic acid. | |
| Coconut | caprylic acid C -8:0 (8%), capric | Coconut |
| | acid, C-10:0, (7%), lauric acid C- | |
| | 12:0, (49%), myristic acid C- | |
| | 14:0(8%), palmitic acid C-16:0 | |
| | (8%), stearic acid C-18:0 (2%), | |
| | oleic acid C-18:1 (6%) and 2% of | |
| | C-18:2 linoleic acid | |
| Corn | 9-12% palmitic acid, 1-3% stearic | Corn germ |
| | acid, 25-35% oleic acid, 40-60% | |
| | linoleic, and trace amouns of lauric | |
| | acid, myristic acid, behenic acid, | |
| | lignoceric acid, erucic acid, and | |
| | palmitoleic acid. | |
| Cottonseed | 52.89% linoleic acid, 25.39% | Cotton |
| | palmitic acid, 16.35% oleic acids, | |
| | together with small amounts of | |
| | 2.33% stearic acid, 1% myristic | |
| | acid, 0.6% palmitoleic acid as well | |
| | as 0.17% linolenic acid | |
| Olive | Oleic acid is the main fatty acid in | Olive |
| | olive oil and accounts for 55-83% | |
| | of total fatty acid content. Olive oil | |
| | also contains variable amounts of | |
| | linoleic acid (3-21%) and linolenic | |
| | acid (<1%). | |
| Palm | Palm oil contains | Palm |
| | approximately 50% saturated fatty | |
| | acids, with 44% palmitic acid | |
| | (C16:0), 5% stearic acid (C18:0), | |
| | and trace amounts of myristic acid | |
| | (C14:0). The unsaturated fatty acids | |
| | are approximately 40% oleic acid | |
| | (C18:1) and 10% polyunsaturated | |
| | linoleic acid (C18:2) and linolenic | |
| | acid (C18:3) | |
| Peanut | oleic acid (45-53%), linoleic acid | Peanut |
| | (27-32%), and palmitic acid (11- | |
| | 14%). | |
| Rapeseed | 60% oleic acid (C18:1), 4% | Rapeseed |
| | palmitic acid (16:0), and 2% stearic | |
| | acid (18:0) | |
| Safflower | Major fatty acids: 55.1-77.0% | Safflower |
| | linoleic acid, 12.45-35.15% oleic | |
| | acid, 5.7-6.81% palmitic acid, and | |
| | 1.88-2.57% stearic acid. | |
| Sesame | The fatty acid composition in | Sesame |
| | sesame seeds consists of abundant | |
| | unsaturated fatty acids: oleic (35.9- | |
| | 42.3%) and linoleic (41.5-47.9%) | |
| | acids from 80% of total fatty acids; | |
| | less than 20% are saturated fatty | |
| | acids, mainly palmitic (7.9-12%) | |
| | and stearic acids (4.8-6.1%) | |
| Sunflower | Sunflower oil contains | Sunflower |
| | approximately 15% saturated, 85% | |
| | unsaturated fatty acid and | |
| | consisting of 14-43% oleic and 44- | |
| | 75% linoleic acids in its unsaturated | |
| | fatty acid content. | |
| Almond | The major fatty acid in almond oil | Almond |
| | is oleic (62.43% in T7-76.34% in | |
| | T4) followed by linoleic (13.97% in | |
| | T4-29.55% in T3) and palmitic | |
| | (4.97% in T2-7.51% inT3) | |
| Beech | The predominant fatty acids present | Beech (Fagus |
| | in triacylglycerols were linoleic | sylvatica L.) |
| | (18:2) and oleic (18:1) followed by | Seed Oil |
| | palmitic (16:0), gadoleic (20:1), | |
| | linolenic (18:3) and stearic (18:0) | |
| | acids in small quantities. | |
| Brazil nut | 0.6% myristic acid, 13.5% palmitic | brazil nut |
| | acid, 0.33% palmitoleic acid, 0.22% | |
| | margaric acid, 11.77% stearic acid, | |
| | 29.9% oleic acid, 42.80% linoleic | |
| | acid, 0.2%0 alpha-linolenic acid, | |
| | 0.54% arachidic acid, 0.21% | |
| | gondoic acid, 0.12% behenic acid, | |
| | 0.34% erucic acid | |
| Cashew | 0.7% myristic acid, 9.93% palmitic | Cashew nut |
| | acid, 0.36% palmitoleic acid, 0.14% | |
| | margaric acid, 8.70% stearic acid, | |
| | 57.24% oleic acid, 20.80% linoleic | |
| | acid, 0.23% alpha-linolenic acid, | |
| | 0.97% arachidic acid, 0.25% | |
| | gondoic acid, 0.39% behenic acid, | |
| | 0.28% erucic acid. | |
| Hazelnut | Fatty acids in hazelnut oil primarily | Hazelnut |
| | consist of oleic acid (73.6%- | (Corylus |
| | 82.6%), linoleic acid (9.8%- | heterophylla) |
| | 16.6%), palmitic acid (4.1%-6.8%) | |
| | and stearic acid (1.6%-3.7%) | |
| Macadamia | oleic acid (C18:1) (45.23-74.86%), | macadamia nut |
| | followed by palmitoleic | |
| | acid (C16:1) (7.81-33.24%), | |
| | palmitic acid (C16:0) (6.44- | |
| | 12.51%), stearic acid (C18:0) | |
| | (1.14-8.51%), and vaccenic acid | |
| | (trans-C18:1) (1.15-4.65%). | |
| Mongongo | elaeostearic acid (18:3) (25%), | Mungongo tree |
| | linoleic acid (18:2) (37%), oleic | (Schinziophyton |
| | acid (18:1) (15%), palmitic and | rautanenii, |
| | stearic acid (18:0) (8-9%, | Euphorbiaceae) |
| | respectively) | |
| Pecan | 4.28% palmitic acid, 0.9% palmitic | pecan nut |
| | acid, 0.10% margaric acid, 1.80% | |
| | stearic acid, 40.63% oleic acid, | |
| | 50.31% linoleic acid, 0.65% alpha- | |
| | linolenic acid, trace amounts of | |
| | arachidic acid, 1.21% gondoic acid, | |
| | 0.16% behenic acid, 0.25% erucic | |
| | acid | |
| Pine Nut | 6.87% palmitic acid, 0.14% | pine nut |
| | palmitoleic acid, 0.10% margaric | |
| | acid, 4.48% stearic acid, 39.55% | |
| | oleic acid, 45.41% linoleic acid, | |
| | 0.63% alpha-linolenic acid, 1.4% | |
| | arachidic acid, 1.6% gondoic acid, | |
| | 0.33% behenic acid, 0.40% erucic | |
| | acid | |
| Pistachio | 0.9% myristic acid, 7.42% palmitic | pistachio nut |
| | acid, 0.70% palmitoleic acid, 0.86% | |
| | stearic acid, 58.19% oleic acid, | |
| | 30.27% linoleic acid, 0.44% alpha- | |
| | linolenic acid, 0.59% arachidic | |
| | acid, 0.60% gondoic acid, 0.34% | |
| | behenic acid, 9.75% erucic acid | |
| Walnut | linoleic acid (60.42-65.77%), oleic | walnut |
| | acid (13.21-19.94%) and linolenic | |
| | acid (7.61-13%) | |
| Pumpkin Seed | The main fatty acids in pumpkin oil | pumpkin seed |
| | seeds are 14.8% palmitic acid (C | |
| | 16:0), 25.8% oleic acid (C18:1), | |
| | and 50.9% linoleic acid (C18:2) | |
| Grapefruit | The main fatty acid components in | grapefruit seed |
| Seed | grapefruit seed oil are palmitic acid | |
| | (27.5-28.9%), Linoleic acid (36.6- | |
| | 39.3%), oleic acid (21.1-25.1%), | |
| | linolenic acid (5.9%), stearic acid | |
| | (2.1-2.9%), and myristic acid (0.8- | |
| | 1.2%) | |
| Lemon | 18.8% palmitic, 0.8% | Citrus limon |
| | heptadecanoic, 3.5% stearic, 30.1% | (lemon) seed oil |
| | oleic, 33.4% linoleic, 13.5% | |
| | linolenic, 0.3% arachidic, 0.3% | |
| | eicosenoic, 0.8% behenic, 0.2% | |
| | lignoceric | |
| Orange | 14-22% palmitic, 2-6% stearic, 26- | Citrus |
| | 35% oleic, 35-45% linoleic, 2-6% | aurantium |
| | linolenic, 0.5% arachidic | dulcis (orange) |
| | | seed oil |
| Watermelon | the main fatty acids in watermelon | watermelon |
| Seed | seed oil are linoleic (59.64%), oleic | seed |
| | (18.7%), palmitic (11.30%), and | |
| | stearic (10.24%) | |
| Acai | palmitic acid 23.0%, palmitoleic | acai pulp |
| | acid 5%, stearic acid 1.3%, oleic | |
| | acid 54.4%, linoleic 16.0% and, | |
| | alpha-linolenic 0.8% | |
| Black cumin | The major fatty acids were linoleic | black cumin |
| Seed | acid (50.2%), oleic acid (19.9%), | (Nigella sativa |
| | margaric acid (10.3%), cis-11,14- | L.) seeds |
| | eicosadienoic acid (7.7%) and | |
| | stearic acid (2.5%) | |
| Blackcurrant | The main fatty acid components of | black currant |
| | black currant seed oil are linoleic | seed |
| | (47.5%), alpha-linolenic (14.5%), | |
| | oleic (13.3%), gamma-linolenic | |
| | (12.6%), palmitic (5.6%), | |
| | stearidonic (2.7%), and stearic | |
| | (1.4%) | |
| Borage | Fatty acid composition of 37.9% | borage seeds |
| | linoleic acid, 24.6% gamma- | (Borago |
| | linolenic acid, 14.8% oleic acid, | officinalis) |
| | 10.2% palmitic acid, 3.3% stearic | |
| | acid, and 0.2% of alpha-linolenic | |
| | and arachidic acids. | |
| Evening | Fatty acid composition of 71.6% | evening |
| Primrose | linoleic acid, 12.6% gamma- | primrose e |
| | linolenic acid, 6.6% oleic acid, | (Oenothera |
| | 5.8% palmitic acid, 2.1% stearic | biennis) |
| | acid, 0.3% arachidic acid, and 0.2% | |
| | alpha-linolenic acid | |
| Flaxseed | The main fatty acid components of | flaxseed |
| | flaxseed oil are alpha-linolenic acid | |
| | (39.9-60.42%), oleic acid (13.44- | |
| | 19.39%), linoleic acid (12.25- | |
| | 17.44%), palmitic acid (4.9-8.0%), | |
| | and stearic acid (2.24-4.59%). | |
| Amaranth | The major fatty acid composition in | Amaranthus |
| | amaranth oil is 12.3-25.9% palmitic | species grain oil |
| | acid, 0.7-4.7% stearic acid, 14.8- | |
| | 38.9% oleic acid, and 33.6-55.9% | |
| | linoleic acid. | |
| Apricot | apricot kernel oil contains 60.01- | apricot kernel |
| | 70.56% oleic acid, 19.74-23.52% | |
| | linoleic acid, 2.35-5.97% palmitic | |
| | acid, 0.8-1.5% stearic acid, and | |
| | 0.2-0.9% palmitoleic acid. | |
| Apple Seed | The main fatty acid components of | apple seed |
| | apple seed oil are linoleic acid | |
| | (50.7-51.4%), oleic acid (37.49- | |
| | 38.55%), palmitic acid (6.51- | |
| | 6.60%), stearic acid (1.75-1.96%) | |
| | and arachidic acid (1.49-1.54%) | |
| Argan | Oleic (43-49%), Linoleic (29- | argan seed |
| | 36$%), Palmitic (11-15%), Stearic | |
| | (4-7%), Palmitoleic (0.3-3%), | |
| | Arachidic (<0.5%), Linolenic | |
| | (<0.2%), Behenic (<0.2%), and | |
| | Myristic (<0.1%) | |
| Avocado | oleic fatty acid (47.2%), followed | avocado seed |
| | by palmitic (23.6%), linoleic | |
| | (13.4%), docosadienoic (8.88%), | |
| | palmitoleic (3.58%), linolenic | |
| | (1.60%), eicosenoic (1.29%), and | |
| | myristic acids (0.33%) | |
| Babssu | The molar percentage for the fatty | babssu oil |
| | acids of babassu oil were caproic | |
| | 0.3, caprylic 7.1, capric 8.3, lauric | |
| | 47.3, myristic 14.5, palmitic 7.1, | |
| | stearic 2.0, arachidic 0.1, oleic 12.2, | |
| | and linoleic acid 1.1 | |
| Ben | The main fatty acid composition of | Moringa |
| | Ben oil is 73.6% oleic acid, 6.25% | oleifera seed |
| | palmitic acid, 6.2% behenic acid, | |
| | 4.97% stearic acid, 3.23% arachidic | |
| | acid, 1.81% gadoleic acid, and | |
| | 1.37% palmitoleic acid | |
| Borneo | Palmitic acid 18.0-22.0%, Stearic | Borneo tallow |
| tallow nut | acid 39-44%, and Oleic acid 38-42, | (tengkawang |
| | % | tallow) is |
| | | derived from |
| | | Shorea |
| | | stenoptera, a |
| | | plant growing in |
| | | the East Indies |
| | | and Malaysia |
| Cape Chestnut | 23.8% palmitic acid, 4.5% stearic | Calodendrum |
| | acid, 33.7% oleic acid, 35.6% | capense seed |
| | linoleic acid, 1.4% linolenic acid, | kernel oil |
| | 1.0% arachidic acid, and trace | |
| | amounts of behenic acid and | |
| | myristic acid. | |
| Carob Pod | Primary fatty acid composition of | carob |
| | carob seed oil is oleic | (Ceratonia |
| | (45.0 ± 0.42%), linoleic | siliqua L.) seed |
| | (32.4 ± 0.30%), palmitic | |
| | (16.6 ± 0.15%), and stearic | |
| | (4.7 ± 0.15%) | |
| Cocoa Butter | 23-30% palmitic acid, 32-37% | cocoa beans |
| | stearic acid, 30-37% oleic acid, | |
| | 2-4% linoleic acid | |
| Cocklebur | fatty acid composition of cocklebur | cocklebur seeds |
| | seeds is 5.59% palmitic acid, 2.14% | |
| | stearic acid, 20.7% oleic acid, | |
| | 68.6% linoleic acid, 0.29% alpha- | |
| | linolenic acid, and 0.18% gamma- | |
| | linolenic acid. | |
| Cohune | 7.5% caprylic, 6.5% capric, 46.5% | Orbignya |
| | lauric, 16% myristic, 9.5% palmitic, | cohune seed oil |
| | 3% stearic, 10% oleic, and 1% | (Cohune) |
| | linoleic | |
| Date Seed | 0.2-0.8% capric acid, 6.6-35.31% | date seed |
| | lauric acid, 0.4-19.3% myristic acid, | |
| | 9.6-13.1% palmitic acid, 0.8-5.67% | |
| | stearic acid, 0.9-1.66% palmitoleic, | |
| | 31.5-52.5% oleic acid, 0.16-.65% | |
| | gadoleic acid, 4.4-12.2% linoleic | |
| | acid, and 0.9-1.68% linolenic acid, | |

TABLE 3
Typical Saturated Fatty Acids Present in Triglycerides.

| | | | Lipid |
|---|---|---|---|
| Common Name | Chemical Name | Structural Formula | Numbers |
| Propionic acid | Propanoic acid | CH₃CH₂COOH | C3:0 |
| Butyric acid | Butanoic acid | CH₃(CH₂)₂COOH | C4:0 |
| Valeric acid | Pentanoic acid | CH₃(CH₂)₃COOH | C5:0 |
| Caproic acid | Hexanoic acid | CH₃(CH₂)₄COOH | C6:0 |
| Enanthic acid | Heptanoic acid | CH₃(CH₂)₅COOH | C7:0 |
| Caprylic acid | Octanoic acid | CH₃(CH₂)₆COOH | C8:0 |
| Pelargonic acid | Nonanoic acid | CH₃(CH₂)₇COOH | C9:0 |
| Capric acid | Decanoic acid | CH₃(CH₂)₈COOH | C10:0 |
| Undecylic acid | Undecanoic acid | CH₃(CH₂)₉COOH | C11:0 |
| Lauric acid | Dodecanoic acid | CH₃(CH₂)₁₀COOH | C12:0 |
| Tridecylic acid | Tridecanoic acid | CH₃(CH₂)₁₁COOH | C13:0 |
| Myristic acid | Tetradecanoic acid | CH₃(CH₂)₁₂COOH | C14:0 |
| Pentadecylic acid | Pentadecanoic acid | CH₃(CH₂)₁₃COOH | C15:0 |
| Palmitic acid | Hexadecanoic acid | CH₃(CH₂)₁₄COOH | C16:0 |
| Margaric acid | Heptadecanoic acid | CH₃(CH₂)₁₅COOH | C17:0 |
| Stearic acid | Octadecanoic acid | CH₃(CH₂)₁₆COOH | C18:0 |
| Nonadecylic acid | Nonadecanoic acid | CH₃(CH₂)₁₇COOH | C19:0 |
| Arachidic acid | Eicosanoic acid | CH₃(CH₂)₁₈COOH | C20:0 |
| Heneicosylic acid | Heneicosanoic acid | CH₃(CH₂)₁₉COOH | C21:0 |
| Behenic acid | Docosanoic acid | CH₃(CH₂)₂₀COOH | C22:0 |
| Tricosylic acid | Tricosanoic acid | CH₃(CH₂)₂₁COOH | C23:0 |
| Lignoceric acid | Tetracosanoic acid | CH₃(CH₂)₂₂COOH | C24:0 |
| Pentacosylic acid | Pentacosanoic acid | CH₃(CH₂)₂₃COOH | C25:0 |
| Cerotic acid | Hexacosanoic acid | CH₃(CH₂)₂₄COOH | C26:0 |
| Carboceric acid | Heptacosanoic acid | CH₃(CH₂)₂₅COOH | C27:0 |
| Montanic acid | Octacosanoic acid | CH₃(CH₂)₂₆COOH | C28:0 |
| Nonacosylic acid | Nonacosanoic acid | CH₃(CH₂)₂₇COOH | C29:0 |
| Melissic acid | Triacontanoic acid | CH₃(CH₂)₂₈COOH | C30:0 |
| Hentriacontylic | Hentriacontanoic | CH₃(CH₂)₂₉COOH | C31:0 |
| acid | acid | | |
| Lacceroic acid | Dotriacontanoic acid | CH₃(CH₂)₃₀COOH | C32:0 |
| Psyllic acid | Tritriacontanoic acid | CH₃(CH₂)₃₁COOH | C33:0 |
| Geddic acid | Tetratriacontanoic | CH₃(CH₂)₃₂COOH | C34:0 |
| | acid | | |
| Ceroplastic acid | Pentatriacontanoic | CH₃(CH₂)₃₃COOH | C35:0 |
| | acid | | |
| Hexatriacontylic | Hexatriacontanoic | CH₃(CH₂)₃₄COOH | C36:0 |
| acid | acid | | |
| Heptatriacontylic | Heptatriacontanoic | CH₃(CH₂)₃₅COOH | C37:0 |
| acid | acid | | |
| Octatriacontylic | Octatriacontanoic | CH₃(CH₂)₃₆COOH | C38:0 |
| acid | acid | | |
| Nonatriacontylic | Nonatriacontanoic | CH₃(CH₂)₃₇COOH | C39:0 |
| acid | acid | | |
| Tetracontylic acid | Tetracontanoic acid | CH₃(CH₂)₃₈COOH | C40:0 |

TABLE 4
Typical Monounsaturated Fatty Acids Present in Triglycerides

| | | | Lipid |
|---|---|---|---|
| | | | Numbers |
| | | | C-Atoms: |
| Common | Chemical | Molecular | Double |
| Name | Name | Formula | Bonds |
| Undecylenic | cis-10-undecenoic acid | C₁₀H₁₉COOH | 11:1 |
| Myristoleic | cis-9-tetradecenoic acid | C₁₃H₂₅COOH | 14:1 |
| Palmitoleic | cis-9-hexadecenoic acid | C₁₅H₂₉COOH | 16:1 |
| Palmitelaidic | trans-9-hexadecenoic acid | C₁₅H₂₉COOH | 16:1 |
| Petroselinic | cis-6-octadecenoic acid | C₁₇H₃₃COOH | 18:1 |
| Oleic | cis-9-octadecenoic acid | C₁₇H₃₃COOH | 18:1 |
| Elaidic | trans-9-octadecenoic acid | C₁₇H₃₃COOH | 18:1 |
| Vaccenic | cis-11-octadecenoic acid | C₁₇H₃₃COOH | 18:1 |
| Gondoleic | cis-9-eicosenoic acid | C₁₉H₃₇COOH | 20:1 |
| Gondolic | cis-11-eicosenoic acid | C₁₉H₃₇COOH | 20:1 |
| Cetoleic | cis-11-docosenoic acid | C₂₁H₄₁COOH | 22:1 |
| Erucic | cis-13-docosenoic acid | C₂₁H₄₁COOH | 22:1 |
| Nervonic | cis-15-tetracosaenoic acid | C₂₃H45COOH | 24:1 |

TABLE 5
Omega-3 Fatty Acids.

| | | Lipid |
|---|---|---|
| Common name | Chemical name | Numbers |
| Hexadecatrienoic | all-cis 7,10,13- | 16:3 (n-3) |
| acid (HTA) | hexadecatrienoic acid | |
| Alpha-linolenic acid (ALA) | all-cis-9,12,15- | 18:3 (n-3) |
| | octadecatrienoic acid | |
| Stearidonic acid (SDA) | all-cis-6,9,12,15,- | 18:4 (n-3) |
| | octadecatetraenoic acid | |
| Eicosatrienoic acid (ETE) | all-cis-11,14,17- | 20:3 (n-3) |
| | eicosatrienoic acid | |
| Eicosatetraenoic acid (ETA) | all-cis-8,11,14,17- | 20:4 (n-3) |
| | eicosatetraenoic acid | |
| Eicosapentaenoic acid (EPA, | all-cis-5,8,11,14,17- | 20:5 (n-3) |
| Timnodonic acid) | eicosapentaenoic acid | |
| Heneicosapentaenoic | all-cis-6,9,12,15,18- | 21:5 (n-3) |
| acid (HPA) | heneicosapentaenoic acid | |
| Docosapentaenoic | all-cis-7,10,13,16,19- | 22:5 (n-3) |
| acid (DPA, Clupanodonic | docosapentaenoic acid | |
| acid) | | |
| Docosahexaenoic | all-cis-4,7,10,13,16,19- | 22:6 (n-3) |
| acid (DHA, Cervonic acid) | docosahexaenoic acid | |
| Tetracosapentaenoic acid | all-cis-9,12,15,18,21- | 24:5 (n-3) |
| | tetracosapentaenoic acid | |
| Tetracosahexaenoic | all-cis-6,9,12,15,18,21- | 24:6 (n-3) |
| acid (Nisinic acid) | tetracosahexaenoic acid | |

TABLE 6
Omega-6 Fatty Acids

| | | Lipid |
|---|---|---|
| Common name | Chemical name | Numbers |
| Linoleic acid (LA) | all-cis-9,12- | 18:2 (n-6) |
| | octadecadienoic acid | |
| Gamma-linolenic acid (GLA) | all-cis-6,9,12- | 18:3 (n-6) |
| | octadecatrienoic acid | |
| Eicosadienoic acid | all-cis-11,14- | 20:2 (n-6) |
| | eicosadienoic acid | |
| Dihomo-gamma-linolenic | all-cis-8,11,14- | 20:3 (n-6) |
| acid (DGLA) | eicosatrienoic acid | |
| Arachidonic acid (AA) | all-cis-5,8,11,14- | 20:4 (n-6) |
| | eicosatetraenoic acid | |
| Docosadienoic acid | all-cis-13,16- | 22:2 (n-6) |
| | docosadienoic acid | |
| Adrenic acid (AdA) | all-cis-7,10,13,16- | 22:4 (n-6) |
| | docosatetraenoic acid | |
| Docosapentaenoic acid | all-cis-4,7,10,13,16- | 22:5 (n-6) |
| (Osbond acid) | docosapentaenoic acid | |
| Tetracosatetraenoic acid | all-cis-9,12,15,18- | 24:4 (n-6) |
| | tetracosatetraenoic acid | |
| Tetracosapentaenoic acid | all-cis-6,9,12,15,18- | 24:5 (n-6) |
| | tetracosapentaenoic acid | |

Conjugated fatty acids could also be used alone or in mixtures with other fatty acids to create triglycerides.

TABLE 7
Conjugated Fatty Acids.

| Common name | Chemical name | Lipid Number |
|---|---|---|
| Rumenic acid | 9Z,11E-octadeca-9,11-dienoic acid | 18:2 (n-7) |
| | 10E,12Z-octadeca-10,12-dienoic acid | 18:2 (n-6) |
| α-Calendic acid | 8E,10E,12Z-octadecatrienoic acid | 18:3 (n-6) |
| β-Calendic acid | 8E,10E,12E-octadecatrienoic acid | 18:3 (n-6) |
| Jacaric acid | 8Z,10E,12Z-octadecatrienoic acid | 18:3 (n-6) |
| α-Eleostearic acid | 9Z,11E,13E-octadeca-9,11,13-trienoic acid | 18:3 (n-5) |
| β-Eleostearic acid | 9E,11E,13E-octadeca-9,11,13-trienoic acid | 18:3 (n-5) |
| Catalpic acid | 9Z,11Z,13E-octadeca-9,11,13-trienoic acid | 18:3 (n-5) |
| Punicic acid | 9Z,11E,13Z-octadeca-9,11,13-trienoic acid | 18:3 (n-5) |
| Rumelenic acid | 9E,11Z,15E-octadeca-9,11,15-trienoic acid | 18:3 (n-3) |
| α-Parinaric acid | 9E,11Z,13Z,15E-octadeca-9,11,13,15-tetraenoic acid | 18:4 (n-3) |
| β-Parinaric acid | all trans-octadeca-9,11,13,15-tetraenoic acid | 18:4 (n-3) |
| Bosseopentaenoic acid | 5Z,8Z,10E,12E,14Z-eicosapentaenoic acid | 20:5 (n-6) |

TABLE 8
Common Triglycerides

Tripropionin: (AKA: Glycerol tripropionoate):
Tributyrin: (AKA: Glyceryl tributurate)
Trivelerin: (Glycerol triverlerate)
Tricaproin: (AKA: Trihexanoin, glycerol trihexanoate)
Tripelargonin: (AKA: Trinonanoin, Glyceryl pelargonate)
Triheptanoin: (AKA: Glyceryl triheptanoate)
Tricaprin: (AKA: Trioctanoin, Tricaprilin, Glycerol trioctanoate)
Tricaprin: (AKA: Tridecaoin, Glycerol tridcanoate, Glycerol tricaprate)
Triundecylin: (AKA: Triundecanoin, Glycerol triundecanoate)
Trilaurin: (AKA: Glycerol trilaurate, Glycerol tridodecanoate)
Tritridecanoin: (AKA: glyceryl tridecanoate)
Trimyristin: (AKA: Glycerol trimysristate)
Tritridecanoin: (AKA: 2,3-di(pentadecanoyloxy)propyl
pentadecanoate tripentadecanoin)
Tripalmatin: (AKA: Glycerol tripalmitate) formulations.
Tripalmitolein: (AKA: Glyceryl tripalmitoleate)
Trimargarin: (AKA triheptadecanoin)
Tristearin: (AKA: Glyceryl tristearate)
Triolein: (AKA: Glyceryl trioleate)
Triheptylundecanoin: (AKA: 2,3-bis(2-heptylundecanoyloxy)propyl 2-
heptylundecanoate)
Trinonadecanoylglycerol: (AKA: Trinonadecanoylglycerol)
Triarachidin: (AKA: Glyceryl triarachidate)
Trierucin: (AKA: Glycerol trieucate)
Tribehenin (AKA: Tridocosanoin)
Tritricosanoin: (AKA Glyceryl tritricosanoate)
Tripentacosylin
Tricerotin
Tricarocerin: (AKA: 2,3-Di(heptacosanoyloxy)propyl heptacosanoate)
Trimontanin: (AKA: Propane-1,2,3-triyl trioctacosanoate)
Trimelissin: (AKA: 2,3-Di(triacontanoyloxy)propyl triacontanoate)
Trihentriacontylin:
Trilacceroin: (AKA: 2,3-Di(dotriacontanoyloxy)propyl dotriacontanoate)
Tripsyllin:
Trigeddin: (AKA: 2,3-Di(tetratriacontanoyloxy)propyl tetratriacontanoate)
Tricerplastin
Trihexatriacontylin
Triheptatria:contylin
Trioctatriacontylin
Trinonatriacontlyin
Tritetracontylin
Triisopalmitin
Triisostearin
Trilinolein
Triricinolein

### Excipients

According to certain embodiments, small concentrations of other excipients can be added to supplement or improve stability or act as an antioxidant. Naturally sourced triglycerides will contain unsaturated moieties and can polymerize and oxidize in the presence of oxygen. α-tocopherols can be added to the triglyceride prior to sterile filtration to act as an antioxidant without impacting emulsion stability or elution characteristics. Other hydrophobic antioxidants such as lycopene, retinols, carotenoids, and other tocopherols can be used.

Similarly, hydrophilic antioxidants such as ascorbic acid can be added to protect hydrophilic APIs. Previous iterations of the formulation have used sodium hydroxide to maintain a desired pH of ˜8 in order to control elution of the API from the lipid excipients. It is imagined that other salts or weak acids could be used to adjust and maintain the formulation at a desired pH. One skilled in the art will know to adjust the buffering agents, if present, to ensure a stable pH is maintained over time.

In certain embodiments, glycerol is utilized as an effective aqueous modifier to produce a stable emulsion. According to further embodiments, sorbitols, manitols, and other polyol chemical compounds are utilized to modify the formulation sufficiently to obtain similar results.

According to certain embodiments, hyaluronic acid, hyaluron, and sodium hyaluronate (collectively referred to herein as “HA”) are considered the same compound for this description and is composed of a long chain polymer containing linear glycosaminoglycan (GAG). One skilled in the art will know that hyaluronic acid can vary in molecular weight (MW) and changes in the MW used will impact formulation viscosity and emulsion stability. In certain embodiments, hyaluronic acid is used as an emulsifier and aqueous phase thickener. The higher the concentration, the higher the lipid to aqueous phase ratio that can be achieved and thus a higher total dose of API available for elution. In some embodiments, HA may be cross-linked with tyraminc.

According to further embodiments, amphipathic polysaccharides that act as a thickener and have both polar hydroxyl and non-polar methyl ether moieties are used to achieve similar effect to HA. In certain embodiments, polyethylene glycol serves as an emulsifier/thickener. In further embodiments, combinations of the foregoing are used to thicken and stabilize the emulsion.

Lecithin, a mixture of phospholipids, obtained from soy or eggs can be used to produce the same result. In certain embodiments, lecithin obtained from other sources such as sunflower seed or canola seed could be used to generate a similar result. Phospholipids, in this instance, is referring to a class of lipids that contain a hydrophilic phosphate group and two hydrophobic fatty acid groups that are joined by an alcohol residue. One skilled in the art will know that the hydrophilic phosphate “head” group can contain different amino acid chemical moieties. Additionally, one skilled in the art will know that the hydrophobic “tail” groups can contain saturated, monounsaturated, and polyunsaturated fatty acids and vary in chain length from 14 to 18 carbons.

In certain embodiments, phospholipids, in combination with other emulsifiers such as, but not limited to, sorbitan esters, polysorbates, propylene oxide, cthoxylates, copolymers, and macromolecules would produce similar results.

In certain embodiments, sufficient aqueous phase thickener is used to achieve a viscosity of the formulation to about 150 cP to about 500 cP. In further embodiments, the increasing presence of anesthetic agent crystals can increase the viscosity of the formulation to about 220 cP.

According to certain embodiments, emulsion components and the range of amount of such components are shown in Table 9.

TABLE 9
Emulsion Stability Summary

| Component | Min | Max |
|---|---|---|
| Soybean Oil % (v/v) | 15 | 80 |
| Medium Chain Triglyceride % (v/v) | 15 | 20 |
| HA % | 0 | 3 |
| Aqueous Buffered to ~8.0 pH | No | Yes |
| Tween 80% (w/v) | 0 | 5 |
| Lecithin % (w/v) | 0 | 5 |
| Glycerol % (w/v) | 1.7 | 2.25 |
| PEG % (w/v) | 0 | 0.5 |
| Sorbitol % (w/v) | 0 | 2 |
| Dextrose % (w/v) | 1 | 2 |
| API (e.g., Ropivacaine) Concentration | 0 | 300 |
| (mg/g_{Soybean oil}) | | |
| NaCl in Aqueous | no | Yes |

### Methods of Formulating Multiphase Stable Emulsion

There are two main methods of manufacturing stable emulsions. The first method utilizes a common solvent, such as acetone (acetone is preferred), other ketone solvents, or mixtures thereof, that can dissolve the triglyceride oil, the lipophilic excipients, and the anesthetic agent. The second method uses hot triglyceride oils to dissolve an effective amount of anesthetic agent.

### Solvent Method

For the solvent manufacturing method, the anesthetic agent and lipophilic excipients are added to a solvent, such as acetone. Once the anesthetic agent is completely dissolved and a clear solution results, the solution can be sterile filtered through a 0.2 micron or smaller pore size filter media. The resulting solution is then subjected to a crystallization unit operation in which the solvent is removed from the solution resulting in the crystallization of the anesthetic agent component in the liquid triglyceride phase. The solvent will be completely removed through various methods, for example the solvent can be removed by pulling vacuum on the solvent phase and exhausting or condensing the solvent to remove it from the liquid lipid phase. Heat may be used to increase the vapor pressure of the solvent, making it easier to remove from the solution. Air, nitrogen, or other inert gases can be used to strip any residual solvent from the liquid lipid phase. Once the solvent is removed from the liquid triglyceride phase, the resulting slurry will contain the anesthetic agent crystals. An aqueous phase containing all remaining emulsifiers and excipients is now sterile filtered through a 0.2 μm filter media and added to the liquid triglyceride phase and the two phases are well mixed to create a homogenous two-phase liquid that is either exposed to a stator-rotor emulsifier or fed to an inline emulsifier to generate a stable homogenous emulsion containing the anesthetic agent. The resulting emulsion is transferred to an inventory tank/bag where it can be packaged into a vial, syringe, or other delivery device. One advantage of using the above solvent method is its ability to dissolve temperature sensitive anesthetic agents into solution without the need for heating. It also allows formation of high concentration anesthetic agent formulations that cannot be made with the heat process.

### Heat Method

The heat manufacturing method utilizes an unexpected and nonobvious method of dissolving the anesthetic agent in the lipid phase, which allows the solution to be sterile filtered before creating the emulsion. Many solvents are able to contain more solute in solution as the solvent is heated but, in most cases, a significant amount of the solute is still present in solution at lower temperature such as room temperature (22° C.). In the example of triglyceride oils, the anesthetic agent is almost completely insoluble in the oil at room temperature but can be increased when the solution is heated. This allows the anesthetic agent in solution to be sterile filtered when heated. Another unique aspect of this formulation is that if it is brought into contact with the aqueous components when heated and allowed to cool as the emulsion is generated, the resulting crystals are smaller and a larger portion of anesthetic agent molecules reside within the oil droplets. Without this hot emulsification, too much of the anesthetic agent is suspended into the formulation as large crystals and the desired elution profile to the target site of several days is not achieved.

In certain implementations, the composition further comprises a radiopaque contrast agent.

Further disclosed herein is a method of treating post-surgical pain in a subject in need thereof comprising administering to the subject and effective amount of a composition comprising stable multiphase emulsion disclosed herein.

In In certain embodiments, the anesthetic is selected from: ambucaine, amolanone, amylcaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dyclonine, ecogonidine, ccogonine, cuprocin, fenalcomine, formocaine, hexylcaine, hydroxyteteracaine, isobutyl p-aminobenzoate, leucinocaine, levoxadrol, lidocaine, mepivacaine, meprylcaine, metabutoxycaine, methyl chloride, myrtecaine, naepaine, octacaine, orthocaine, oxethazaine, parenthoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, or a pharmaceutically acceptable salt thereof, or a mixture thereof. In certain implementations, the anesthetic is ropivacaine. In certain alternative embodiments the anesthetic in bupivacaine.

In certain embodiments, the anesthetic agent is an amide anesthetic. In exemplary implementations the amide anesthetic is articaine, bupivacaine, cinchocaine, etidocaoine, bupivacaine, lidocaine, mepivacaine, prilocaine, ropivacaine, and/or trimecaine.

In certain embodiments, the anesthetic agent is an ether anesthetic. Exemplary ether anesthetics include, but are not limited to benzocaine, chloroprocaine, cocaine, cyclomethycaine, dimethocaine (larocaine), piperocaine, propoxycaine, procaine, proparacaine, and/or tetracaine.

In certain implementations, the anesthetic agent is from a natural source. Exemplary natural source anesthetics include, but are not limited to: saxitoxin, neosaxitoxin, tetrodotoxin, menthol, eudenol, cocaine, spilanthol.

In certain embodiments, the anesthetic agent is combined with one or more antiemetic agents. Exemplary antiemetic agents include but are not limited to NK1 receptor antagonists (e.g., Aprepitant, Casopitant, Rolapitant), cannabinoids (e.g., cannabis, cannabidiol, nabilone, dronabinol, THC), and/or benzodiazepine (e.g., midazolam, lorazepam).

In certain embodiments, the anesthetic agent is combined with one or more vasoconstrictors (e.g., epinephrine).

In certain embodiments, the anesthetic agent is combined with one or more anti-hypertensives: (e.g., clonidine, and/or dexmedetomidine).

In certain embodiments, the anesthetic agent is combined with one or more non-steroidal anti-inflammatory (NSAIDS). Examples include, but are not limited to Salicylates (e.g., Acetylsalicylic acid, Diflunisal, Salicylic acid/salts, Salsalate), propionic acid derivatives (e.g., Ibuprofen, Fenoprofen, flurbiprofen, pelubiprofen, dexibupropfen, ketoprofen, oxaprozin, zaltoprofen, naproxen, dexketoprofen, loxoprofen), acetic acid derivatives (e.g., Indomethacin, sulindac, ketorolac, aceclofenac, tolmetin, etodolac, diclofenac, bromfenac), enolic acid derivatives (e.g., Piroxicam, Tenoxicam, Lornoxicam, Phenylbutazone, Meloxicam, droxicam), Selective COX-2 inhibitors (e.g., Celecoxib, Firocoxib, Parecoxib, Etoricoxib), Sulfonanilides, Nimesulide, Clonixin, and/or Licofelone.

According to still further embodiments, the anesthetic agent is combined with one or more antihistamines/Involuntary nervous system blocker. Examples include but are not limited to: Meclizine, hyoscine, chloropeniramine, and/or diphenylhydramine.

According to still further embodiments, the anesthetic agent is combined with one or more additional agent, including but not limited to: an anti-microbial agent, and anti-inflammatory agent (dexamethasone) and/or a procoagulant agent.

Also provided herein are kits of pharmaceutical formulations containing the disclosed compounds or compositions. The kits may be organized to indicate a single formulation or combination of formulations. The composition may be sub-divided to contain appropriate quantities of the compound. The unit dosage can be packaged compositions such as packeted powders, vials, ampoules, prefilled syringes, or sachets containing liquids.

The compound or composition described herein may be a single dose or for continuous or periodic discontinuous administration. For continuous administration, a kit may include the compound in each dosage unit. For periodic discontinuation, the kit may include placebos during periods when the compound is not delivered. When varying concentrations of the composition, the components of the composition, or relative ratios of the compound or other agents within a composition over time is desired, a kit may contain a sequence of dosage units.

The kit may contain packaging or a container with the compound formulated for the desired delivery route. The kit may also contain dosing instructions, an insert regarding the compound, instructions for monitoring circulating levels of the compound, or combinations thereof. Materials for performing using the compound may further be included and include, without limitation, reagents, well plates, containers, markers or labels, and the like. Such kits are packaged in a manner suitable for treatment of a desired indication. Other suitable components to include in such kits will be readily apparent to one of skill in the art, taking into consideration the desired indication and the delivery route. The kits also may include, or be packaged with, instruments for assisting with the injection/administration or placement of the compound within the body of the subject. Such instruments include, without limitation, syringe, pipette, forceps, measuring spoon, eye dropper or any such medically approved delivery means. Other instrumentation may include a device that permits reading or monitoring reactions in vitro.

The compound or composition of these kits also may be provided in dried, lyophilized, or liquid forms. When reagents or components are provided as a dried form, reconstitution generally is by the addition of a solvent. The solvent may be provided in another packaging means and may be selected by one skilled in the art.

A number of packages or kits are known to those skilled in the art for dispensing pharmaceutical agents. In one embodiment, the package is a labeled blister package, dial dispenser package, or bottle.

Various aspects and embodiments of the present disclosure are defined by the following numbered clauses:
1. A composition for treating post-surgical pain in a subject in need thereof, comprising: an emulsion comprising:
   an aqueous carrier; and liquid lipid phase dispersed into droplets within the aqueous carrier, and a first anesthetic within the lipid phase.
2. The composition of clause 1, wherein the first anesthetic comprises a first plurality of anesthetic crystals.
3. The composition of clause 1, wherein the first anesthetic is dissolved with the lipid phase.
4. The composition of any of clauses 1-3, further comprising a second plurality of anesthetic crystals within the aqueous carrier, but not the lipid phase and wherein the second plurality of anesthetic crystals dissolves and elutes from the emulsion at a faster rate than the first plurality anesthetic crystals.
5. The composition of any of clauses 1-4, wherein the composition further comprises one or more additional anesthetics, different from the first anesthetic.
6. The composition of any of clauses 1-5, wherein the lipid phase comprises a triglyceride.
7. The composition of any of clauses 1-6, wherein the aqueous carrier further comprises a thickener.
8. The composition of clause 7, wherein the thickener is sodium hyaluronate, hyaluronan, and/or hyaluronic acid.
9. The composition of clause 7, wherein the aqueous carrier further comprises a polyol.
10. The composition of clause 9, wherein the polyol is glycerol and is present in an amount of from about 0.25 to about 2.5% (w/v) of the composition.
11. The composition of any of clauses 1-10, wherein composition has a viscosity of from about 150 to about 500 Cp.
12. The composition of clause 11, wherein the composition has a viscosity of from about 200 to about 250 Cp.
13. The composition of any of clauses 1-12, wherein the lipid phase further comprises a phospholipid present in an amount from about 0.1% to about 2.0% of the lipid phase.
14. The composition of any of clauses 1-13, wherein the lipid phase is from about 10% to about 40% (w/v).
15. The composition of any of clauses 1-14, wherein the stable emulsion further comprises lecithin, and wherein lecithin is present in amount of from 0.1-20% (w/v) of the emulsion.
16. The composition of any of clauses 1-15, wherein the emulsion further comprises dextrose, and wherein dextrose is present in amount of from about 1-2% (w/v) of the emulsion.
17. The composition of any of clauses 1-16, wherein the emulsion further comprises sorbitol, and wherein sorbitol is present in amount of from about 0.1-2% (w/v) of the emulsion.
18. The composition of any of clauses 1-17, wherein the lipid phase comprises soybean oil in an amount from about 15% to 80% (v/v) and one or more medium chain triglycerides in amount from about 15% to about 20% (v/v).
19. The composition of clause 18, wherein the anesthetic agent is present in an amount from about 0.1 mg/g soybean oil to about 300 mg/g soybean oil.
20. The composition of any of clauses 1-19, wherein the first anesthetic is selected lidocaine, prilocaine, mepivacaine, ropivacaine, etidocaine, levobupivacaine, bupivacaine, cocaine, procaine, 2-chloroprocaine, tetracaine, benzocaine, amethocaine, chlorocaine, butamben, dibucaine, and ester analogs of aconitine, dyclonine, ketamine, pramoxine, safrole, and salicyl alcohol.
21. The composition of any of clauses 1-20, wherein the lipid phase comprises tributyrate and/or stearate.
22. The composition of clause 2, wherein the lipid phase further comprises an oil and/or wax that is solid at 25° C. in an amount of from about 5% to about 30% of the lipid phase (w/w) and wherein the oil and/or wax coats the anesthetic crystals.
23. The composition of any of clauses 1-22, wherein the emulsion is stable for at least six months.
24. The composition of any of clauses 1-23, wherein the anesthetic is ropivacaine and/or bupivacaine.
25. A composition for treating pain in a subject in need thereof, comprising: an emulsion comprising:
   a lipid carrier phase; and an aqueous phase dispersed into droplets within the lipid carrier phase, and a first anesthetic within the aqueous phase.
26. The composition of clause 25, wherein the first anesthetic comprises a first plurality of anesthetic crystals.
27. The composition of clauses 25 or 26, further comprising a second plurality of anesthetic crystals present within the lipid carrier, but not the aqueous phase, and wherein the second plurality of anesthetic crystals dissolves and elutes from the emulsion at a faster rate than the first plurality anesthetic crystals.
28. The composition of clause 27, wherein the anesthetic is hydrophilic and is dissolved within the aqueous phase.
29. The composition of any of clauses 25-28, wherein the composition further comprises one or more additional anesthetics, different from the first anesthetic.
30. The composition of any of clauses 1-29, wherein the emulsion is stable for a period of about 1 month to about 2 years.
31. The composition of clause 30, wherein the emulsion is stable for a period of about 6 months to about 12 months.
32. The composition of any of clauses 1-29x2, wherein the emulsion is reversible.
33. A method of treating pain in a subject in need thereof comprising administering to the subject and effective amount of a composition comprising:
   an emulsion comprising:
   an aqueous carrier; and lipid phase dispersed into droplets within the aqueous carrier, and a first anesthetic within the lipid phase and wherein the anesthetic is eluted from the composition over a period of between about 4 and about 7 days.
34. The method of clause 33, wherein the anesthetic comprises a plurality of anesthetic crystals, and wherein the composition further comprises a second plurality of anesthetic crystals within the aqueous carrier, but not the lipid phase and wherein the second plurality of anesthetic crystals dissolves and elutes from the emulsion at a faster rate than the first plurality anesthetic crystals.
35. The method of clause 33, wherein the composition is delivered near a nerve or nerve bundle of a subject and wherein the nerve or nerve bundle innervates the surgical incision area of the subject.

### EXPERIMENTAL

The following examples are put forth so as to provide those of ordinary skill in the art with a complete disclosure and description of how the compounds, compositions, articles, devices and/or methods claimed herein are made and evaluated and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Example 1-Preparation of Stable Emulsion

Stable emulsions containing the anesthetic ropivacaine were prepared according to the following protocol:
1. Ropivacaine was added to soy oil. (up to 13% by weight of oil).
2. 0.75 g of lecithin was added to the soy oil mixture.
3. Anesthetic agent/Oil slurry was heated to 100C with mixing the slurry vigorously until the crystals of anesthetic agent are completely dissolved into solution.
4. Soybean oil containing the anesthetic agent was sterile filtered using 0.2 μm vacuum filter at temperature (100C).
5. An aqueous solution of 0.15% sodium and 0.51 g glycerol (w/v) was prepared at 4° C.
6. The aqueous solution was heated to 100° C. and sterile filtered by passing it through a 0.2 um filter media before combining it with the hot soy oil, lecithin, and anesthetic agent base solution.
7. The two phases in the main mixing vessel were emulsified with a rotor-stator emulsifier or by feeding the two-phase mixture to an in line emulsifier until a stable emulsion formed.
8. Emulsification was continued until mixture cooled to room temperature for subsequent use or storage.

### Example 2-Porcine Sciatic Peripheral Nerve Block

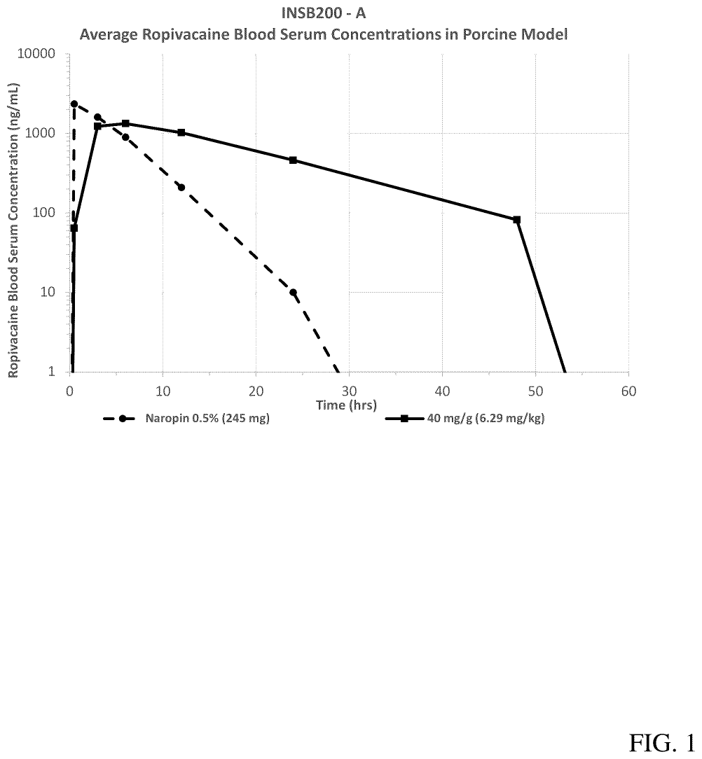

The instantly disclosed stable emulsion was prepared according to the methods of Example 1 as either a 6.29 mg/Kg (40 mg ropivacaine/g lipid) formulation or a 20.44 mg/Kg (130 mg ropivacaine/g lipid dose) formulation. Pigs received injection of either one of the stable emulsion formulations or a Naropin 0.5% positive control into the transfacial space surrounding the sciatic nerve to produce a peripheral nerve block (PNB). Animal behavior was observed and recorded following the injections. FIG. 1 shows the ropivacaine blood plasma concentration over time for a 6.29 mg/Kg formulation. The Naropin line shows standard of care for peripheral nerve block. The stable emulsion formulation provides a significant amount of ropivacaine for greater than 48 hours.

TABLE 10
Behavioral observations for animals that received peripheral nerve
block of sciatic nerve with 40 mg Ropivacaine/g lipid dose.

| | Time Post- | | |
|---|---|---|---|
| Animal | Injection | | Behavioral Observations |
| Animal 1 | 6 | hour | Animal not moving limb |
| | 12 | hour | Slightly uncoordinated; |
| | | | no knuckling observed |
| | 96 | hour | Slight toe tapping |
| | Day 6-Day 14 | | Normal Gait |
| Animal 2 | 6 | hour | Some discoordination |
| | 12 | hour | More coordinated, very |
| | | | slight toe tapping |
| | 24 | hour | Normal Gait |
| | Day 2-Day 14 | | Normal Gait |
| Animal 3 | 6 | hour | Knuckling, but fully motile |
| | 12 | hour | Occasional knuckling, taking |
| | | | some exaggerated steps |
| | 24 | hour | Slight knuckling |
| | Day 2-Day 14 | | Normal Gait |
| Animal 4 | 6 | hour | Slight toe tapping |
| | 12 | hour | Normal Gait |

| 24 hour-Day | Normal Gait |
|---|---|
| 14 | |

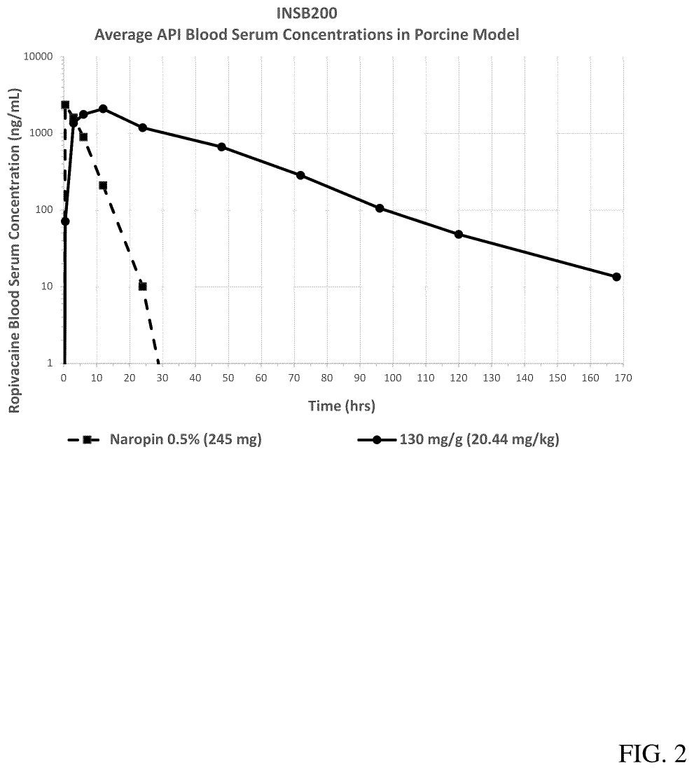

FIG. 2 shows blood plasma concentration of a 20.44 mg/Kg formulation of the stable emulsion drug product. These data show the formulation provides a significant dose of ropivacaine for over 168 hours and suggest a faster onset to PNB. The formulation provides excellent elution of API out beyond 72 hours and likely provides sensory block for greater than 72 hours.

TABLE 11
Behavioral observations for animals that received peripheral nerve
block of sciatic nerve with 130 mg Ropivacaine/g lipid dose.

| | Time Post- | |
|---|---|---|
| Animal | Injection | Behavioral Observations |
| Animal 1 | 6 hour | Knuckling |
| | 12 hour-Day 14 | Normal Gait |
| Animal 2 | 6 hour | No knuckling |
| | 12 hour-Day 14 | Normal gait |
| Animal 3 | 6 hour | Knuckling with some |
| | | uncoordinated motility |
| | 12 hour-Day 14 | Normal Gait |
| Animal 4 | 6 hour | Knuckling but fully motile |
| | 12 hour-Day 14 | Normal Gait |

### Formula Variations

In Examples 3-6, a base formula is used with various alternations made for each example. The base formulation is given below in Table 12. Mixing of components in the base formula is done using a benchtop mixer with a standard 2-inch head at 6000 RPM. This base formulation, and the various alternations in these Examples, are merely illustrative implementations and are not intended to be restrictive.

TABLE 12
Base Formulation

| Material Name | % (w/w) | mg/mL |
|---|---|---|
| API | 3.009 | 29.34 |
| Soybean Oil | 37.62 | 366.8 |
| Glycerin, Natural | 1.743 | 17.000 |
| Lipoid S75 (Lecithin) | 2.564 | 25.000 |
| Sodium Hyaluronate | .082 | 0.8000 |
| Plant Generated Water for injection (WFI) | 54.94 | 561.2 |

### Example 3-Types of Lecithin

As would be understood, lecithin composition changes depending on where it is sourced (plant, animal, egg), whether it is de-oiled, and degree of processing. Lecithin with high percentages of phosphatidylcholine is often used for injectable products and must meet the USP National Formulary requirements for injection including a phosphatidylcholine concentration >70%. Lecithin with lower concentrations of phosphatidylcholine is typically used for oral and topical drug products, food, and cosmetics. The lecithin with lower phosphatidylcholine results in more stable emulsions with lower energy input during formation, but higher phosphatidylcholine concentration Lecithin still form stable emulsions but require higher energy input during formation. In this Example, five samples, samples 1-5, were developed and analyzed using lecithin of differing composition.

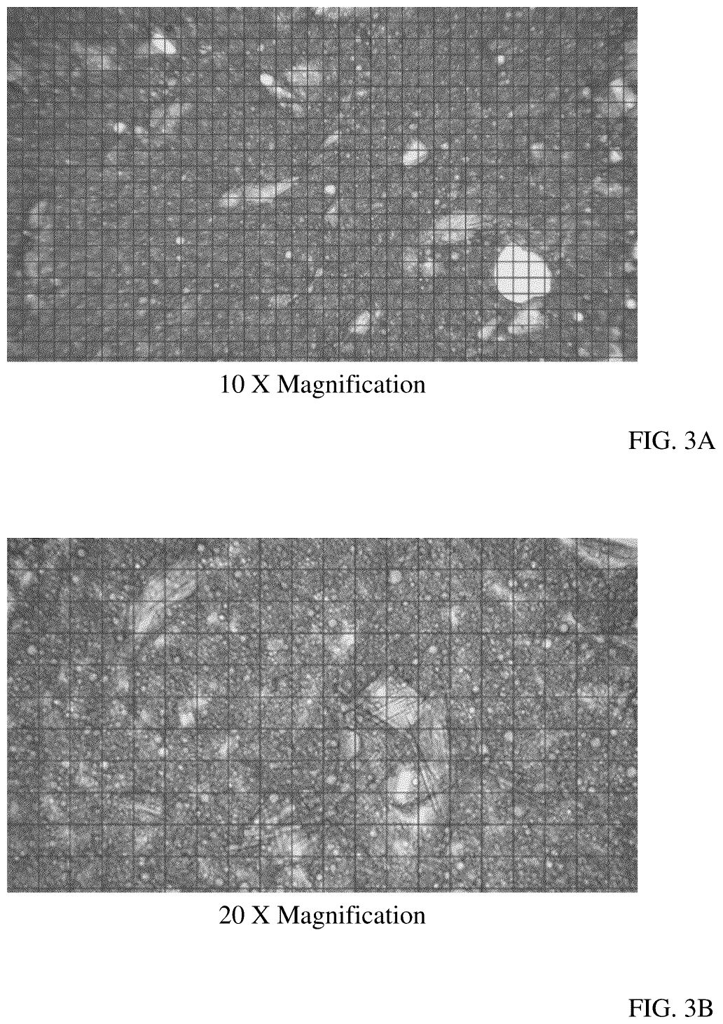

Sample 1 uses the base formulation but with Spectrum NF (50-60% phosphatidylcholine) replacing the lecithin component. Images of the resulting material are shown under 10× magnification in FIG. 3A and under 20× magnification in FIG. 3B. The oil droplets are small, and the API crystals are long needle form. The emulsion remained stable for 14 days. The sample forms a white opaque emulsion/suspension.

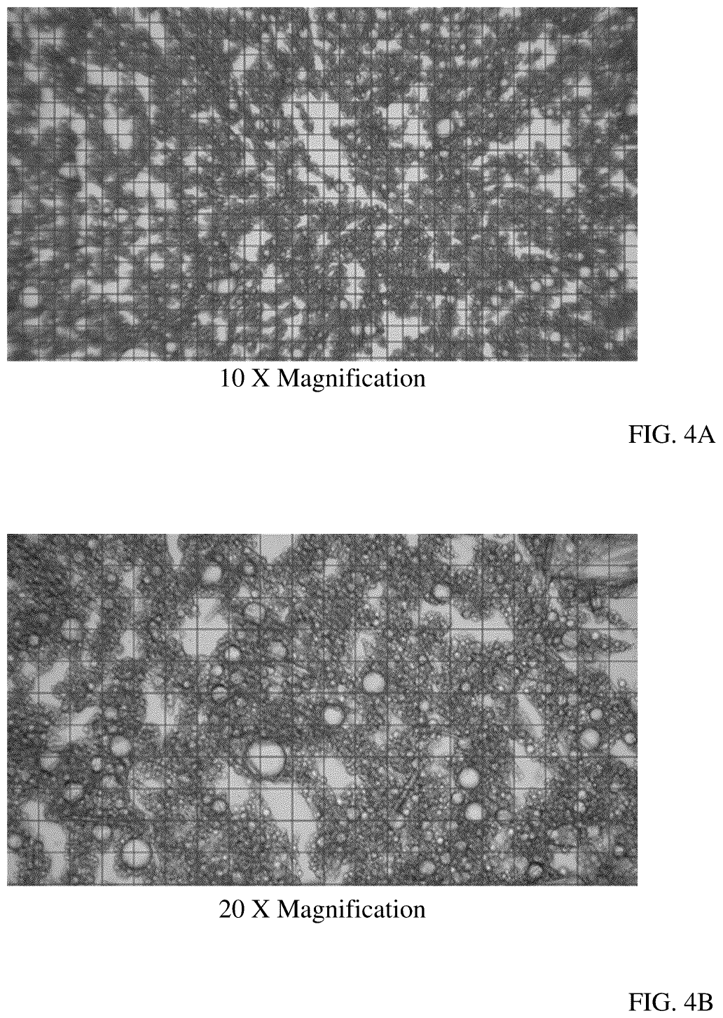

Sample 2 uses the base formulation but with Cargill Metarin Lecithin (19-27% phosphatidylcholine) replacing the lecithin component. Images of the resulting material are shown under 10× magnification in FIG. 4A and under 20× magnification in FIG. 4B. The oil droplets are small, and the API crystals are long needle form. The emulsion remained stable for 1 day. The sample forms a white opaque emulsion/suspension.

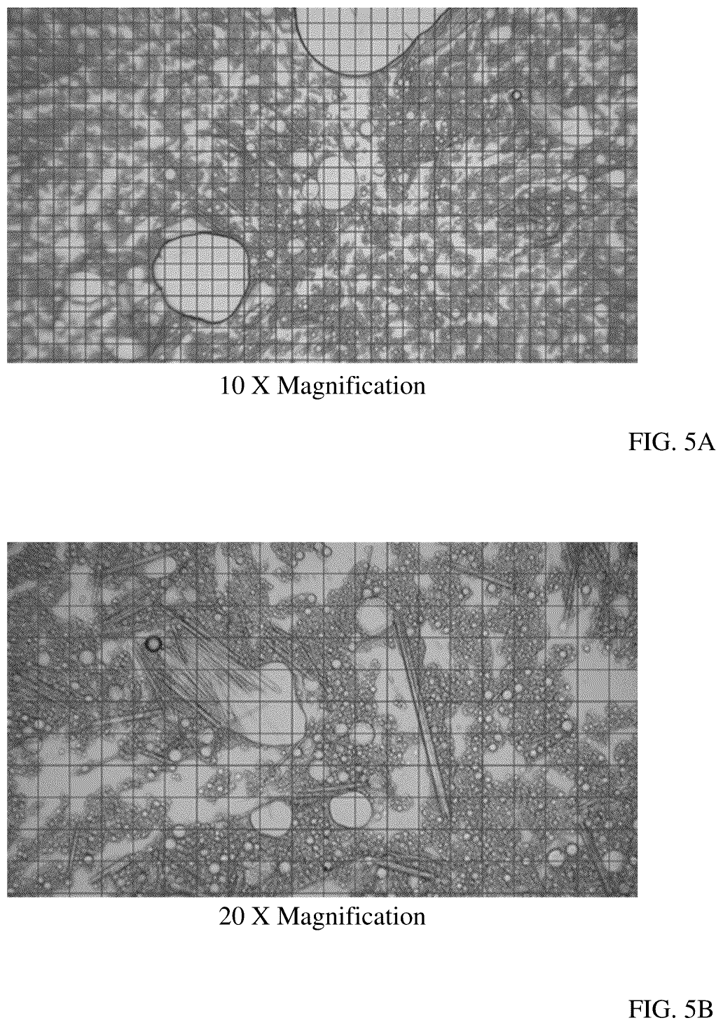

Sample 3 uses the base formulation but with Cargill Epikuron Lecithin (19-27% phosphatidylcholine) replacing the lecithin component. Images of the resulting material are shown under 10× magnification in FIG. 5A and under 20× magnification in FIG. 5B. The oil droplets are small, and the API crystals are long needle form. The emulsion remained stable for 1 day but can be re-suspended or re-emulsified into an acceptable emulsion through agitation, such as shaking. The sample forms a white opaque emulsion/suspension.

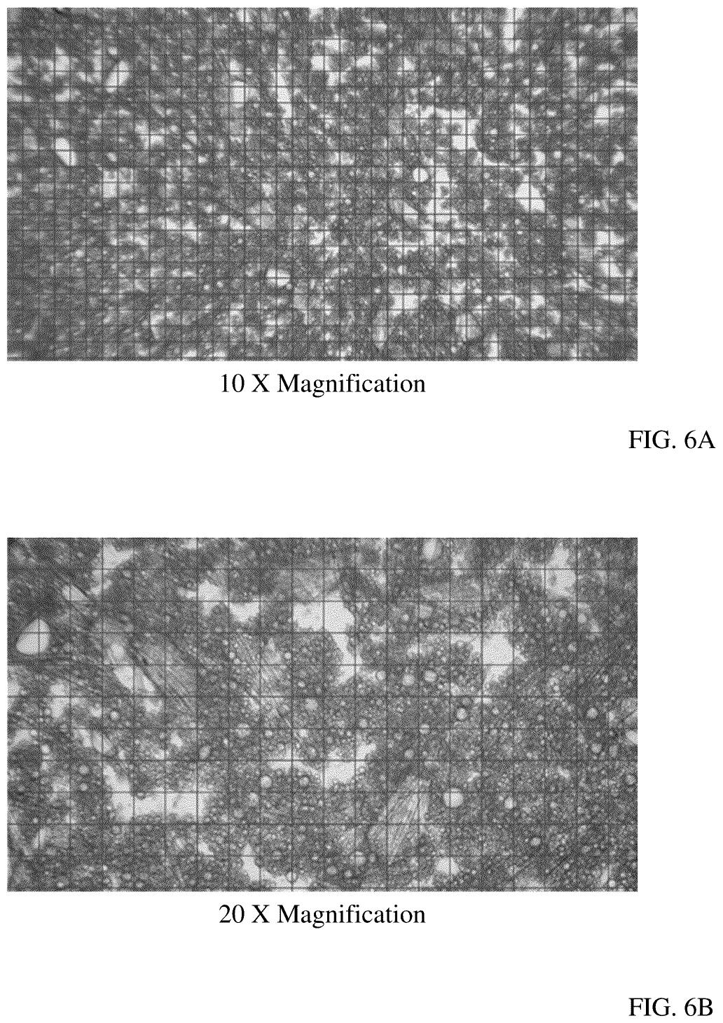

Sample 4 uses the base formulation but with Lipoid S75 (>70% phosphatidylcholine) replacing the lecithin component. Images of the resulting material are shown under 10× magnification in FIG. 6A and under 20× magnification in FIG. 6B. The oil droplets are small, and the API crystals are long needle form. The emulsion remained stable for 4 days but can be re-suspended or re-emulsified into an acceptable emulsion through agitation, such as shaking. The sample forms a white opaque emulsion/suspension.

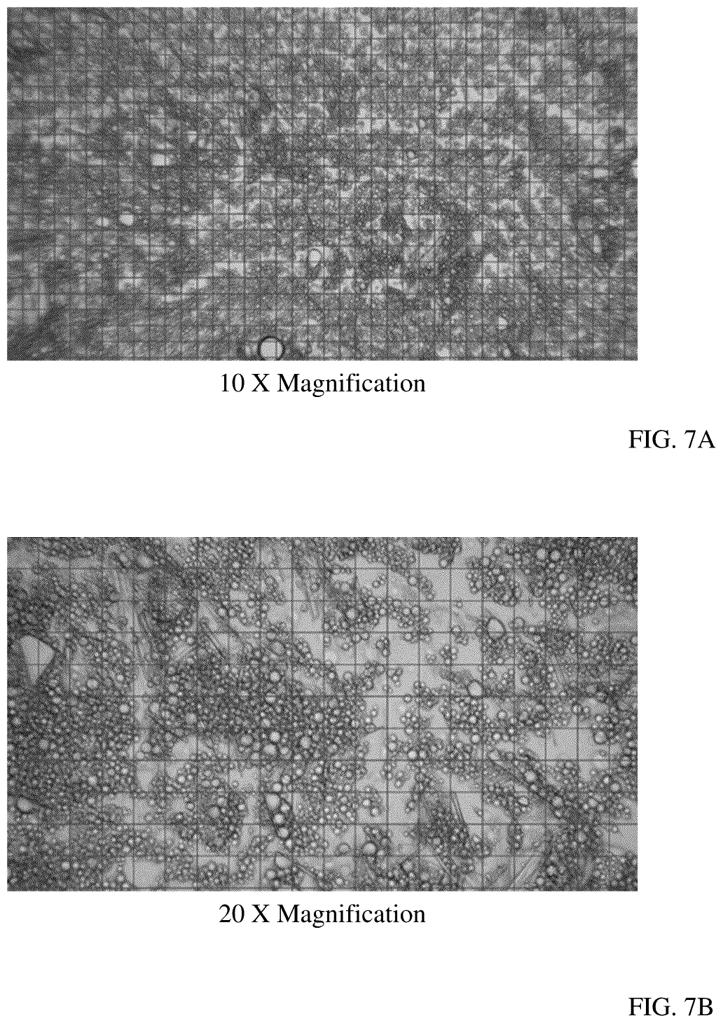

Sample 5 uses the base formulation but with Lipoid S80 (73-79% phosphatidylcholine) replacing the lecithin component. Images of the resulting material are shown under 10× magnification in FIG. 7A and under 20× magnification in FIG. 7B. The oil droplets are small, and the API crystals are long needle form. The emulsion remained stable for 1 day but can be re-suspended or re-emulsified into an acceptable emulsion through agitation, such as shaking. The sample forms a white opaque emulsion/suspension.

### Example 4 Lecithin Concentration Relative to Soybean Oil

Those skilled in the art would understand that lecithin concentration may affect the formulation by changing the interaction between the aqueous and lipid phases at the boundaries between them. Typically, a higher lecithin ratio relative to the lipid phase concentration results in smaller non-continuous phase droplets. However, it may also be the case that the higher lecithin concentration may prevent the API crystals from aggregating. The higher lecithin concentration may also make the formulation very viscous. In the use case of pain therapeutics, the higher viscosity may be acceptable as the delivery needle can be a large gauge needle for placement of the drug product. In this Example, five samples, samples 6-10, were developed and analyzed using differing concentrations of lecithin.

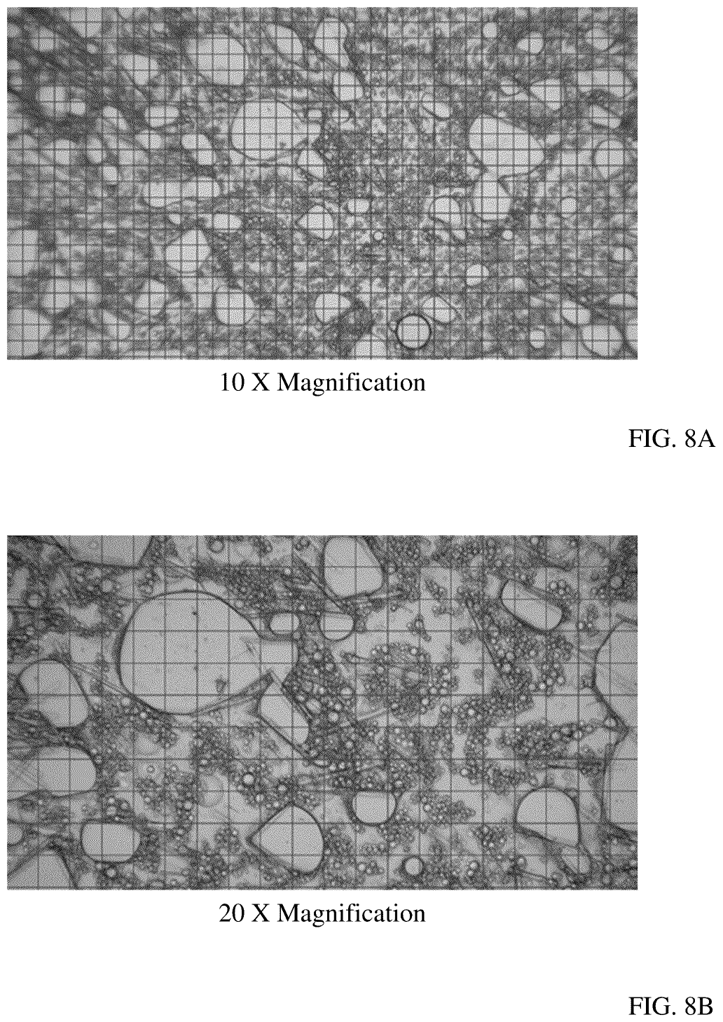

Sample 6 uses the base formulation but with the lecithin component being reduced to 0.5% and replaced with soybean oil. Images of the resulting material are shown under 10× magnification in FIG. 8A and under 20× magnification in FIG. 8B. The oil droplets are small, and the API crystals are long needle form. The emulsion remained stable for 3 days but can be re-suspended or re-emulsified into an acceptable emulsion through agitation, such as shaking. The sample forms a white opaque emulsion/suspension. Note that the large voids in FIGS. 8A and 8B are air pockets entrapped in the sample.

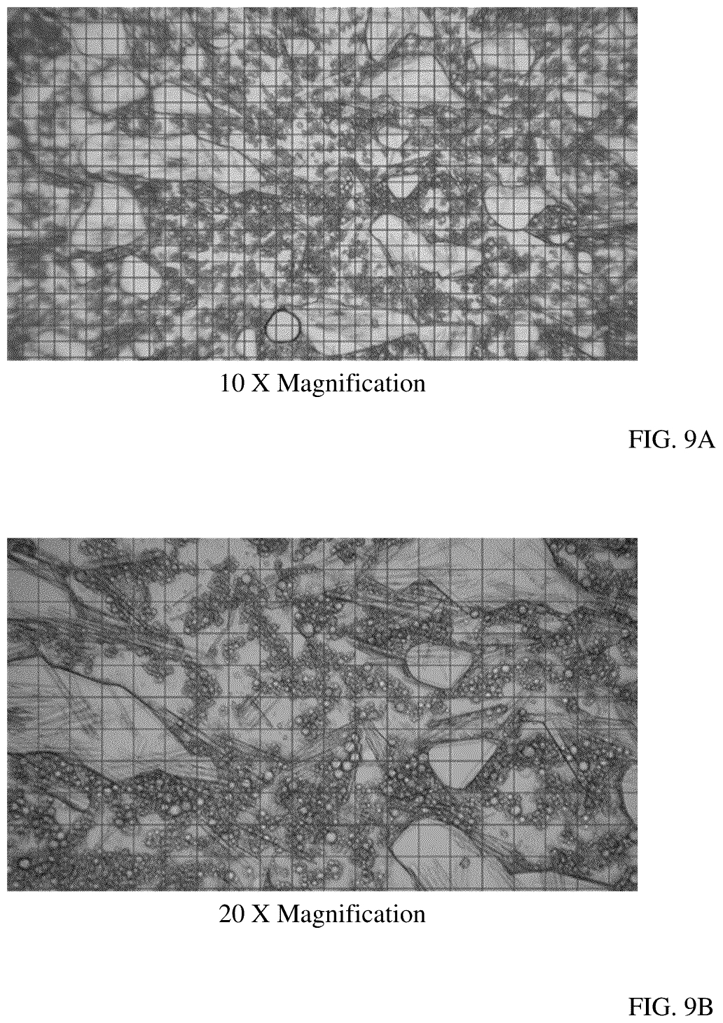

Sample 7 uses the base formulation but with the lecithin component being slightly reduced to 2.5% and replaced with soybean oil. Images of the resulting material are shown under 10× magnification in FIG. 9A and under 20× magnification in FIG. 9B. The oil droplets are small, and the API crystals are long needle form. The emulsion remained stable for 4 days but can be re-suspended or re-emulsified into an acceptable emulsion through agitation, such as shaking. The sample forms a white opaque emulsion/suspension. Note that the large voids in FIGS. 9A and 9B are air pockets entrapped in the sample.

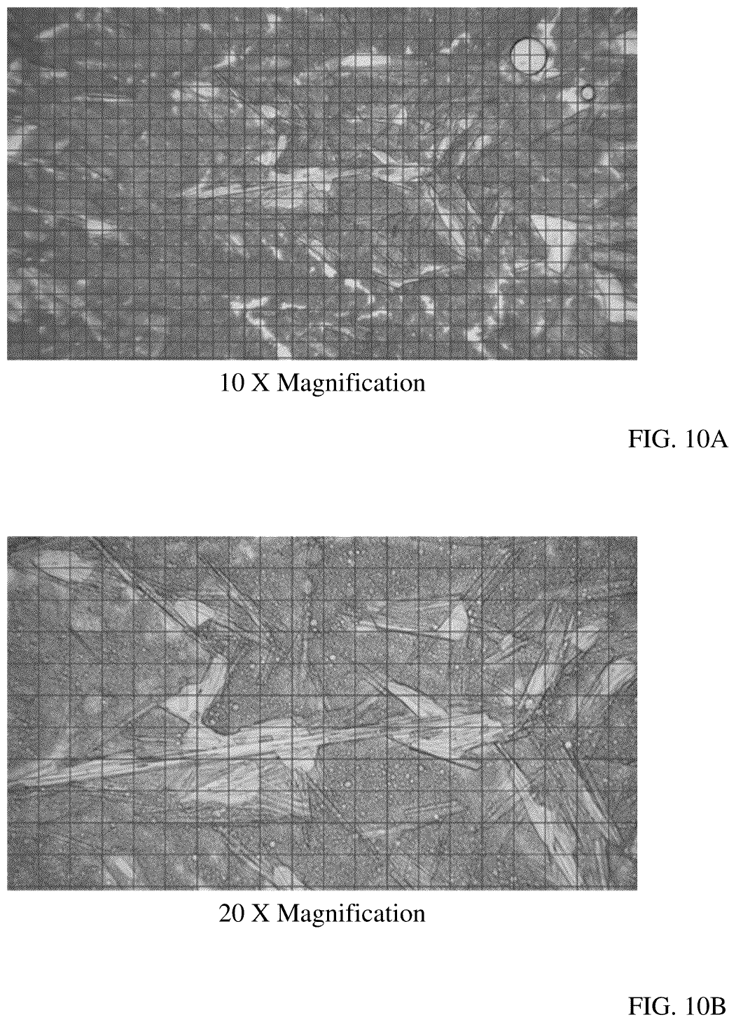

Sample 8 uses the base formulation but with the lecithin component being increased to 5%, with soybean oil being removed to compensate. Images of the resulting material are shown under 10× magnification in FIG. 10A and under 20× magnification in FIG. 10B. The oil droplets are small, and the API crystals are long needle form. The emulsion remained stable for 9 days but can be re-suspended or re-emulsified into an acceptable emulsion through agitation, such as shaking. The sample forms a white opaque emulsion/suspension. Note that the large voids in FIGS. 10A and 10B are air pockets entrapped in the sample.

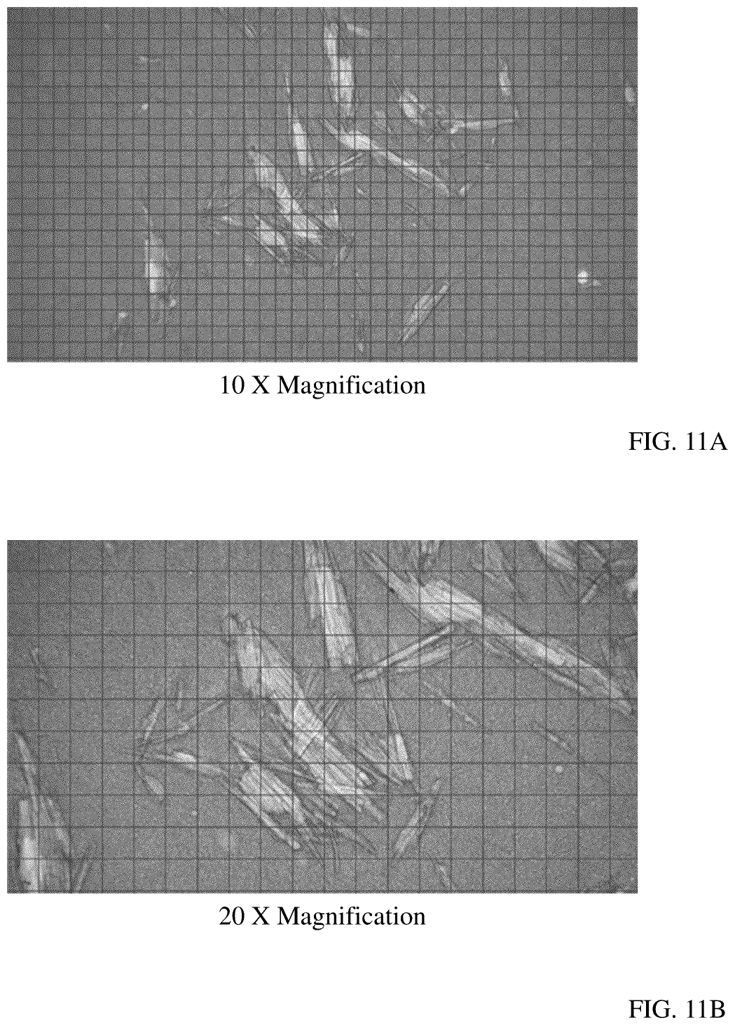

Sample 9 uses the base formulation but with the lecithin component being increased to 10%, with soybean oil being removed to compensate. Images of the resulting material are shown under 10× magnification in FIG. 11A and under 20× magnification in FIG. 11B. The oil droplets are very small, and the API crystals are long needle form. The crystals in the sample have aggregated into clumps. The emulsion remained stable for 23+ days. The sample forms a white opaque emulsion/suspension. Note that the large voids in FIGS. 11A and 11B are air pockets entrapped in the sample.

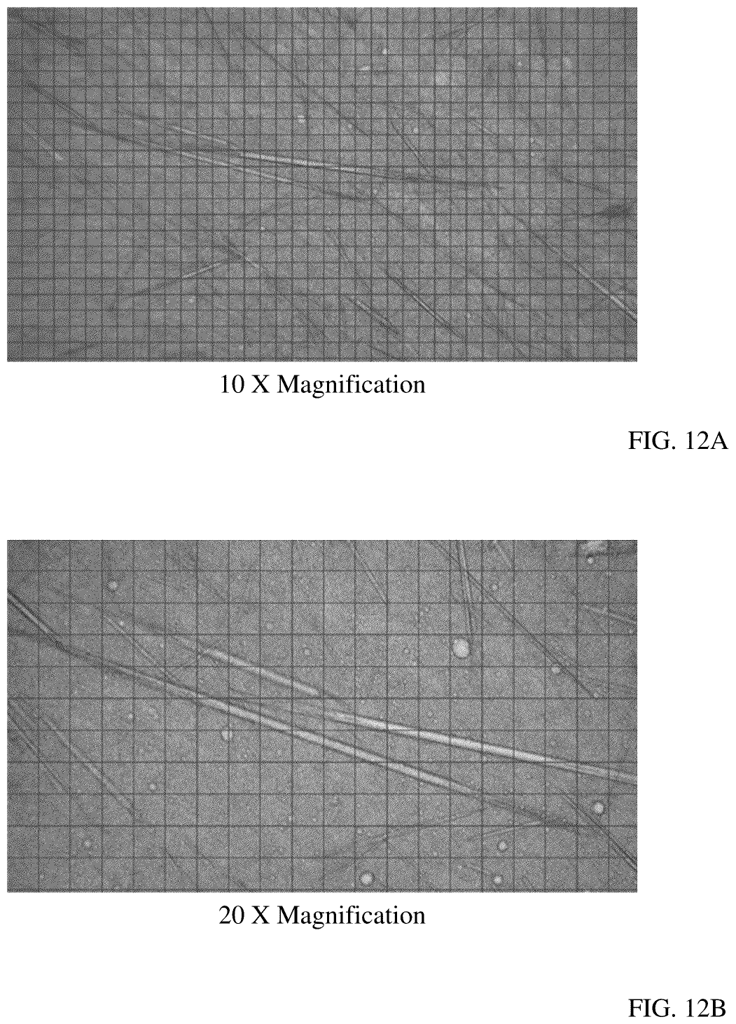

Sample 10 uses the base formulation but with the lecithin component being increased to 20%, with soybean oil being removed to compensate. Images of the resulting material are shown under 10× magnification in FIG. 12A and under 20× magnification in FIG. 12B. The oil droplets are very small, and the API crystals are long needle form. The crystals in the sample are separated from each other and show very little aggregation. The emulsion remained stable for 15 days. The sample forms a white opaque emulsion/suspension that is very viscous, such that it would be difficult to inject.

### Example 5 Aqueous Phase Thickening

In this Example, sodium hyaluronate was used as a thickening agent for the aqueous phase to slow or impede the interaction between the lipid non continuous phase droplets and stabilize the emulsion. Hyaluronic acid, from sodium hyaluronate, may also act as an emulsifier, as the molecule has both polar and nonpolar moieties. In this Example, sodium hyaluronate concentration is measured relative to the aqueous phase, not the whole drug.

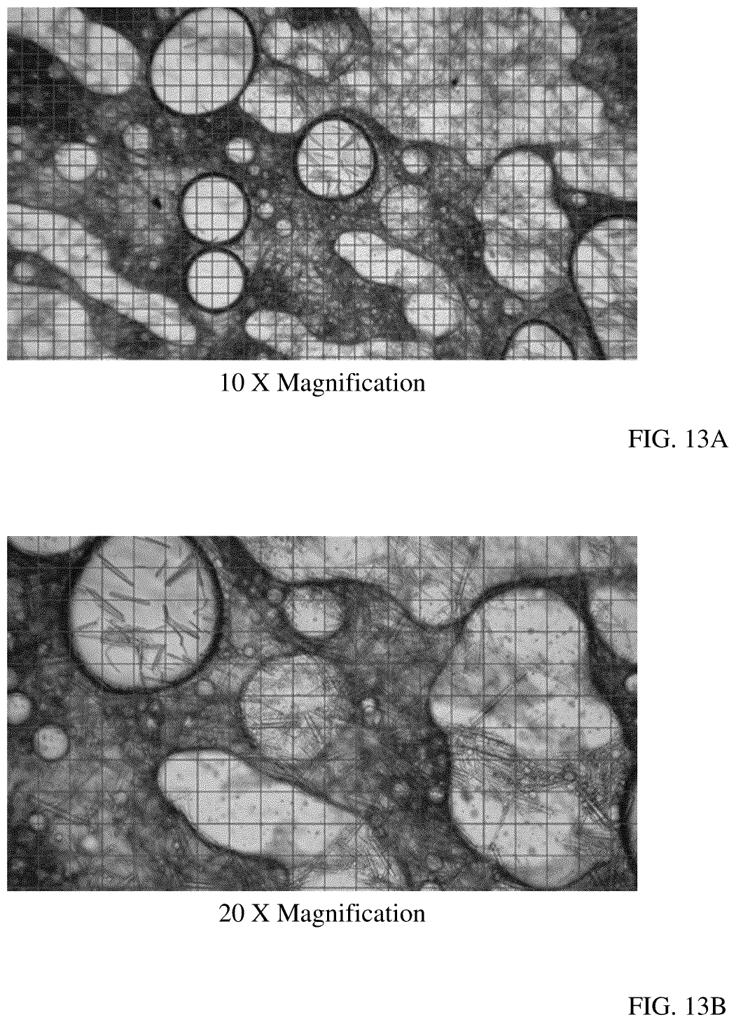

Sample 11 uses the base formulation but has no sodium hyaluronate in the aqueous phase. Images of the resulting material are shown under 10× magnification in FIG. 13A and under 20× magnification in FIG. 13B. The oil droplets are large, and the API crystals are long needle form. The emulsion of the sample broke quickly—within minutes—and separated into oil and aqueous phases.

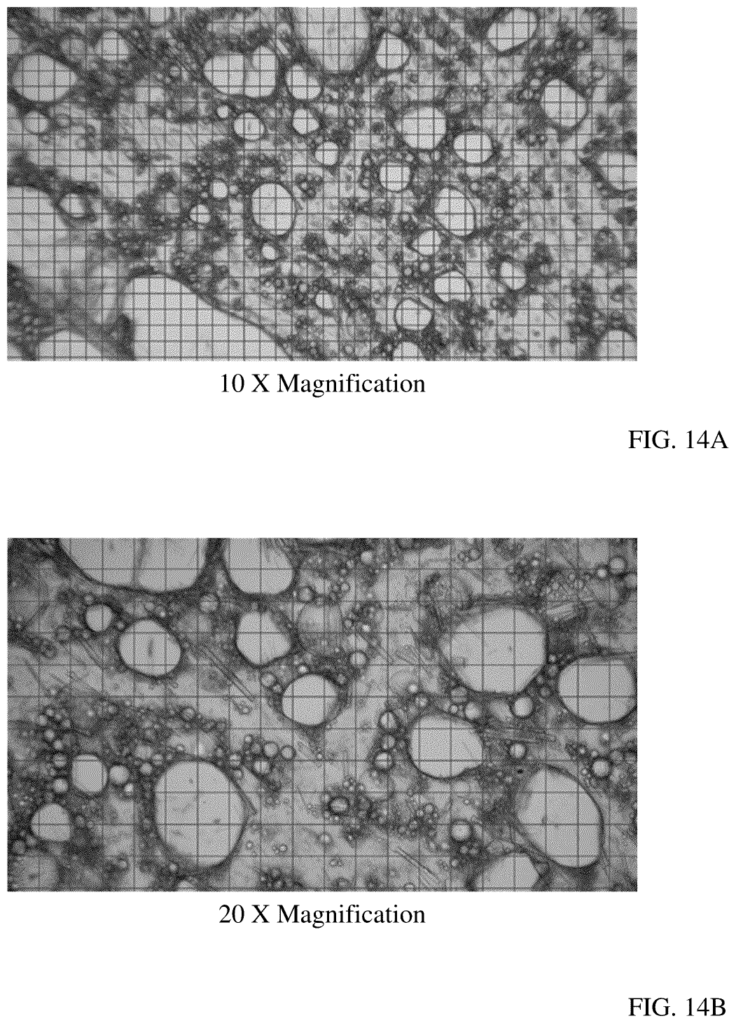

Sample 12 uses the base formulation but has a sodium hyaluronate concentration of 0.1 percent in the aqueous phase. Images of the resulting material are shown under 10× magnification in FIG. 14A and under 20× magnification in FIG. 14B. The oil droplets are smaller than those of sample 11. The API crystals are long needle form. The emulsion remained stable for 2 days but can be re-suspended or re-emulsified into an acceptable emulsion through agitation, such as shaking.

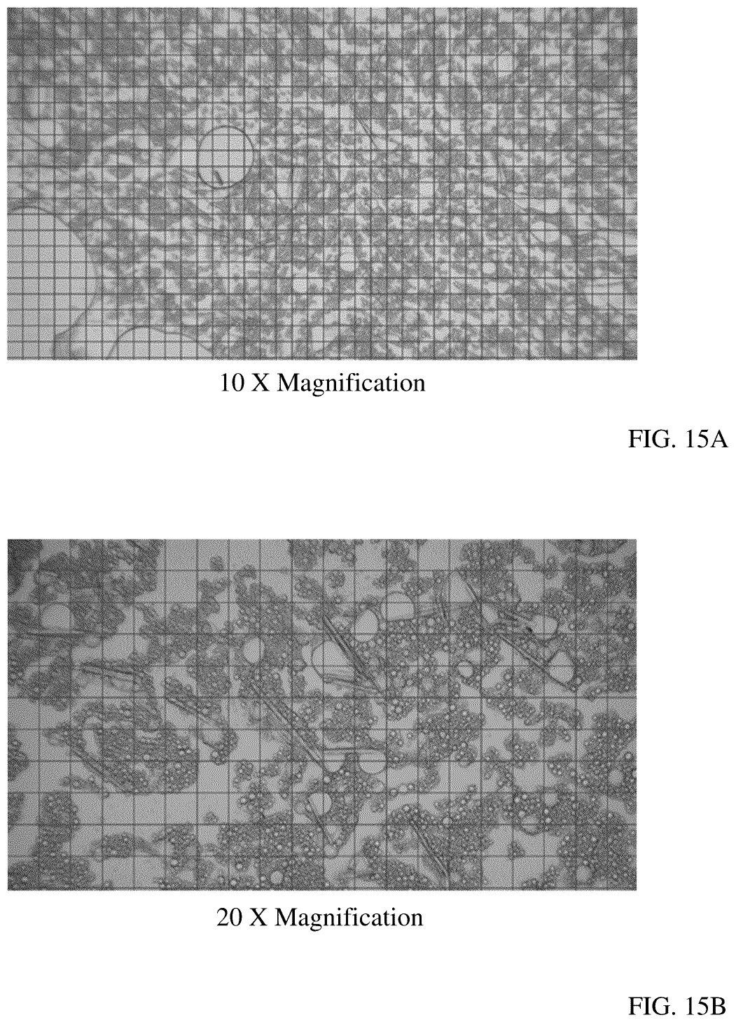

Sample 13 uses the base formulation but has a sodium hyaluronate concentration of 0.15 percent in the aqueous phase. Images of the resulting material are shown under 10× magnification in FIG. 15A and under 20× magnification in FIG. 15B. The oil droplets are small and relatively uniform. The API crystals are long needle form. The emulsion showed increased stability over sample 11.

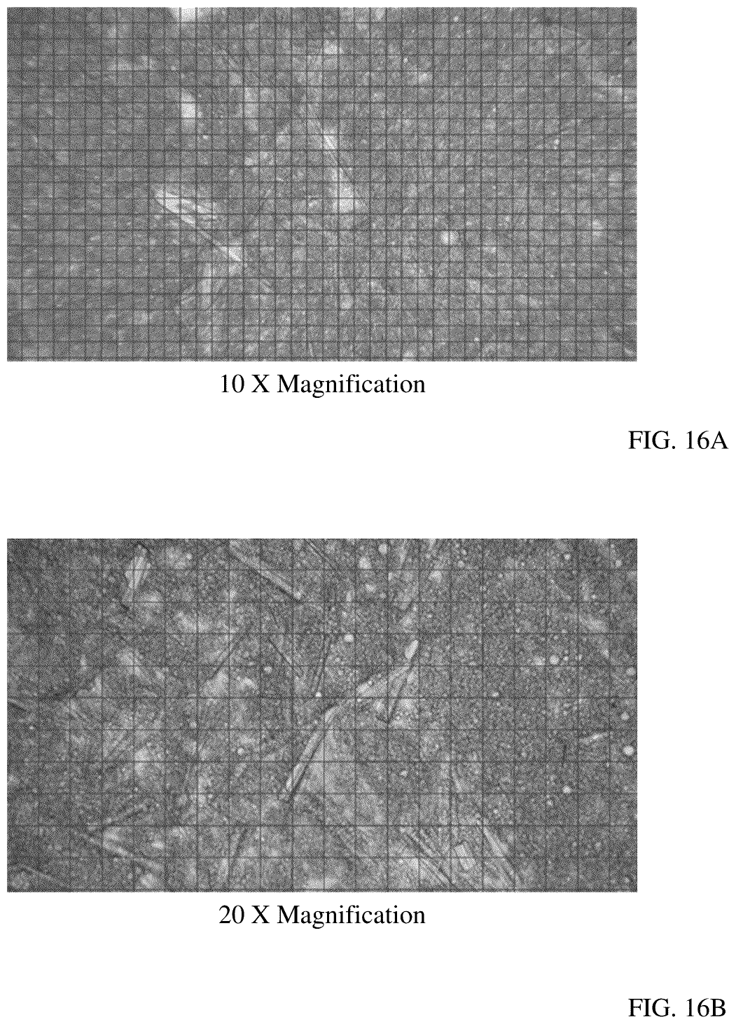

Sample 14 uses the base formulation but has a sodium hyaluronate concentration of 1 percent in the aqueous phase. Images of the resulting material are shown under 10× magnification in FIG. 16A and under 20× magnification in FIG. 16B. The oil droplets are small and relatively uniform. The API crystals are long needle form. The emulsion remained stable for 8 days.

As can be seen, at concentrations below 0.1 percent, the emulsions quickly broke and separated. At concentrations above 0.1 percent, the emulsions became progressively more stable.

### Example 6 Addition of Glycerol

In this Example, glycerol was added as an aqueous phase modifier for the aqueous phase, which tended to increase the viscosity of the aqueous phase. Glycerol concentration in this Example is measured relative to the whole formulation, rather than any particular phase.

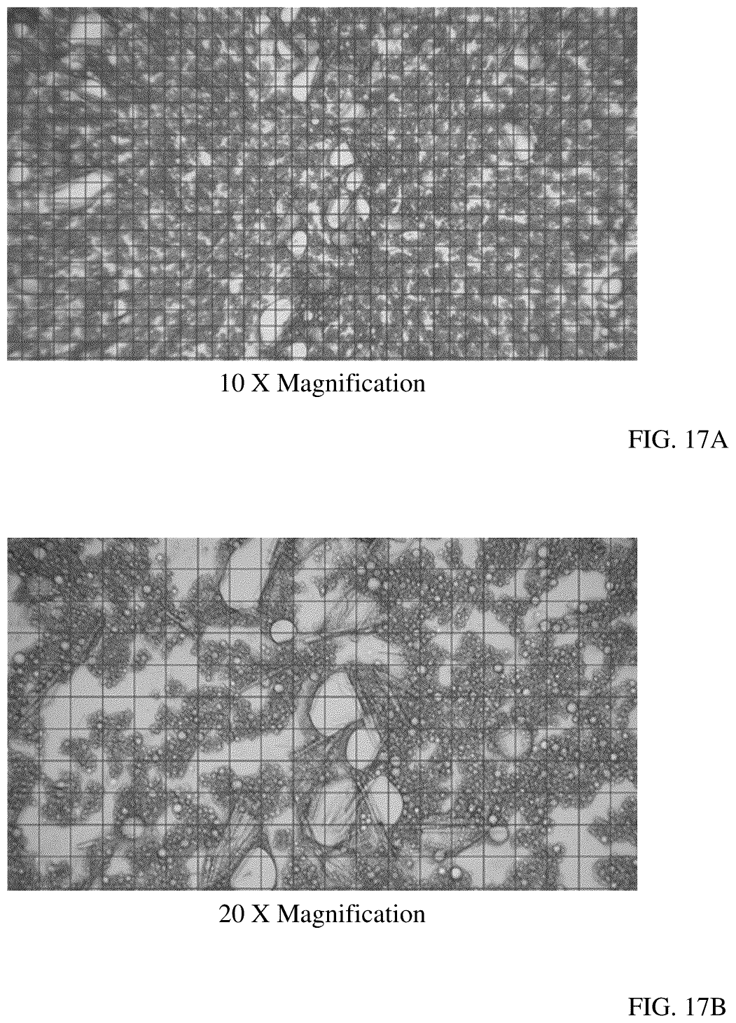

Sample 15 uses the base formulation but has a glycerol concentration of 1.7% relative to the whole formulation. Images of the resulting material are shown under 10× magnification in FIG. 17A and under 20× magnification in FIG. 17B. The oil droplets are small and relatively uniform. The API crystals are long needle form. The emulsion remained stable for 1 day.

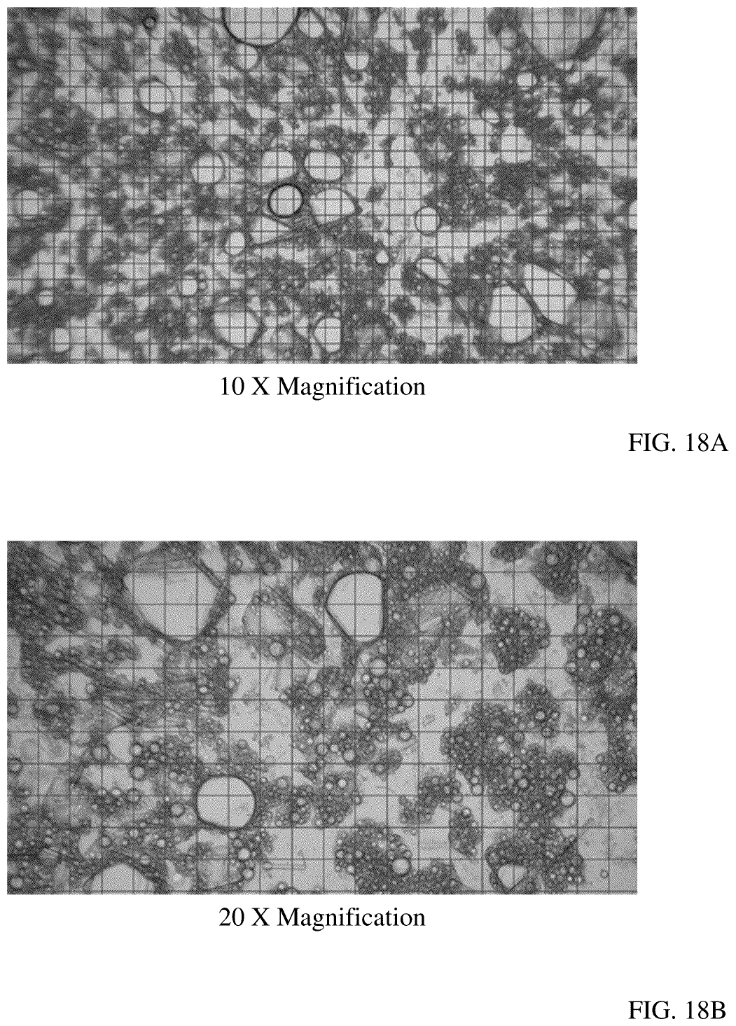

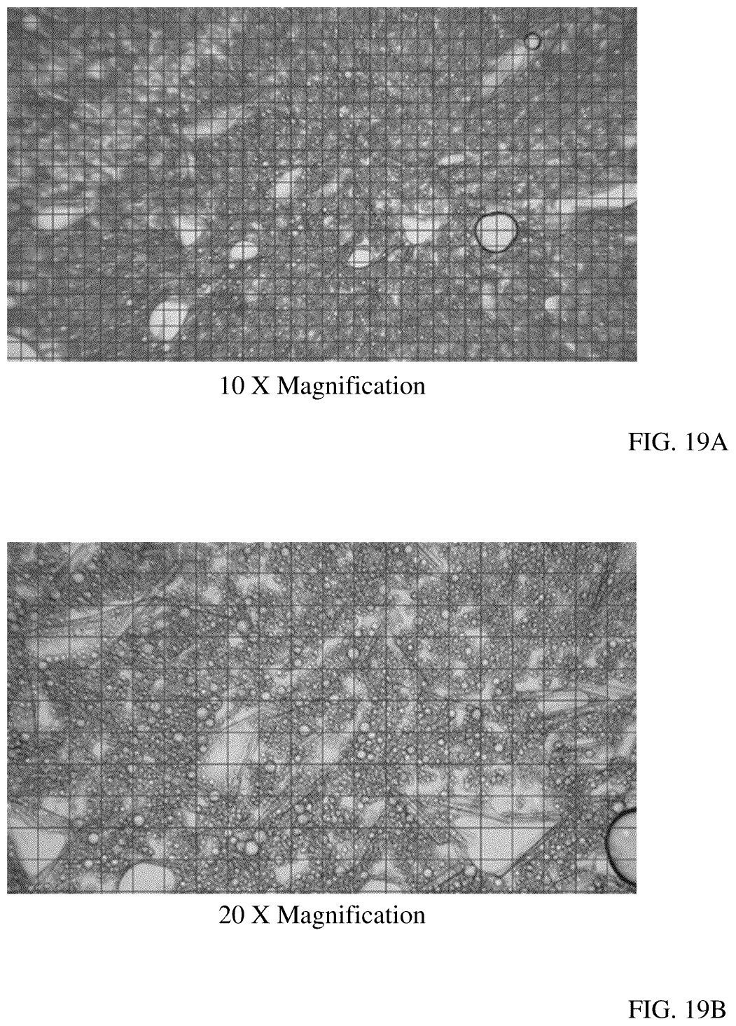

Sample 16 uses the base formulation but has a glycerol concentration of 3.0% relative to the whole formulation. Images of the resulting material are shown under 10× magnification in FIG. 18A and under 20× magnification in FIG. 19B. The oil droplets are small and relatively uniform. The API crystals are long needle form. The emulsion remained stable for 1 day.

Sample 17 uses the base formulation but has a glycerol concentration of 5.0% relative to the whole formulation. Images of the resulting material are shown under 10× magnification in FIG. 19A and under 20× magnification in FIG. 19B. The oil droplets are small and relatively uniform. The API crystals are long needle form. The emulsion remained stable for 1 day.

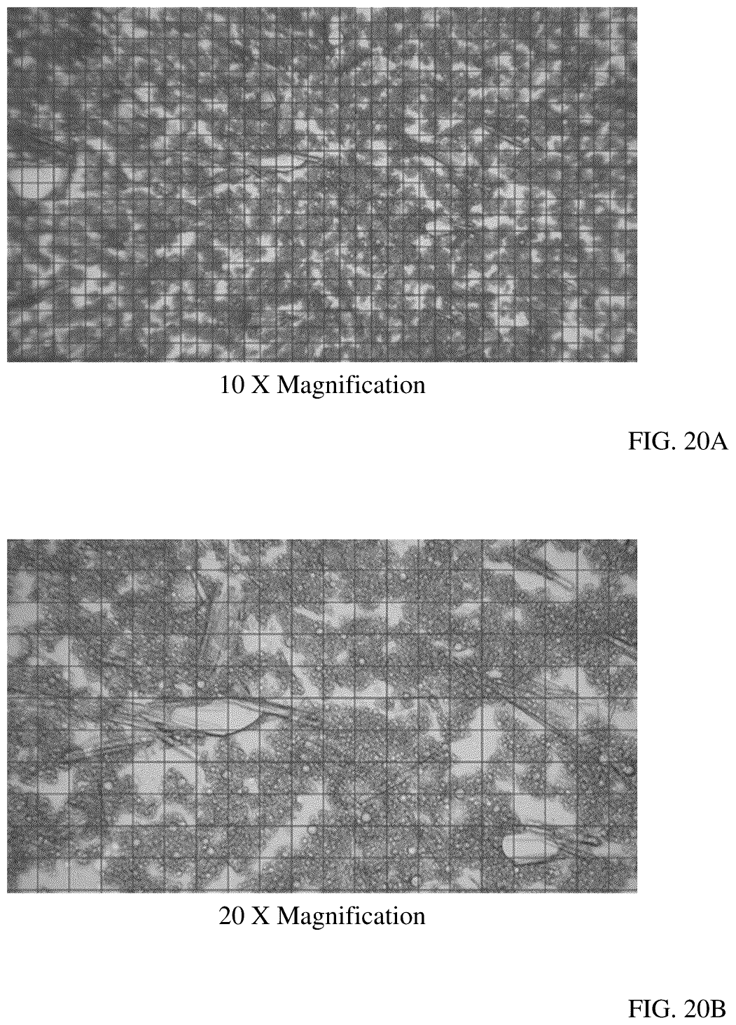

Sample 18 uses the base formulation but has a glycerol concentration of 10% relative to the whole formulation. Images of the resulting material are shown under 10× magnification in FIG. 20A and under 20× magnification in FIG. 20B. The oil droplets are smaller and more uniform than samples 18, 19, and 20. The API crystals are long needle form. The emulsion remained stable for 1 day.

As sample 18 showed improvements in oil drop size and consistency, a positive correlation between the concentration of glycerol or a similar solvent and the droplet consistency. A negative correlation can be seen between glycerol/solvent concentration and droplet size.

### Example 7 Lipid Phase Thickening

In this Example, either coconut oil or carnauba wax was added to the lipid phase of the formulation to increase its viscosity. As the thickening agent, coconut oil or carnauba wax, is added to the formulation, the added mass of soy oil was equally reduced to maintain the total amount of lipid phase in the formulation. Thickening agent concentrations in this Example are measured relative to the whole formulation, rather than any particular phase.

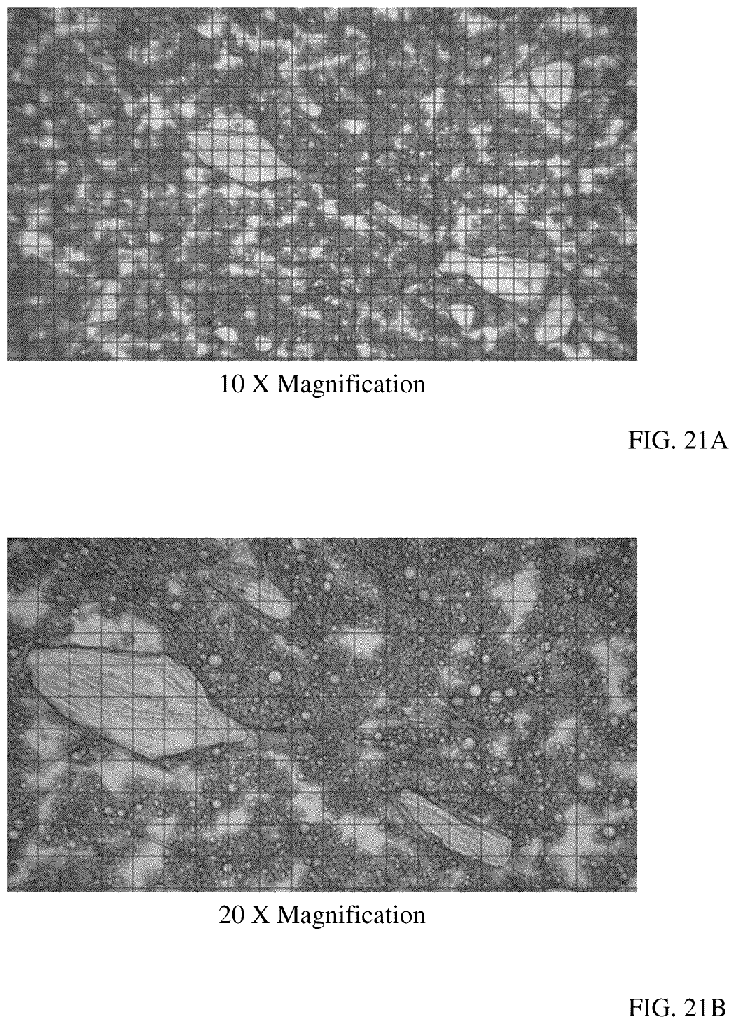

Sample 19 uses the base formulation but with 10% of the formulation consisting of coconut oil and an equal reduction in soy oil. Images of the resulting material are shown under 10× magnification in FIG. 21A and under 20× magnification in FIG. 21B. The oil droplets are generally small, but an increase in the size and number of large droplets can be observed. The API crystals are long needle form. The product was a stable emulsion.

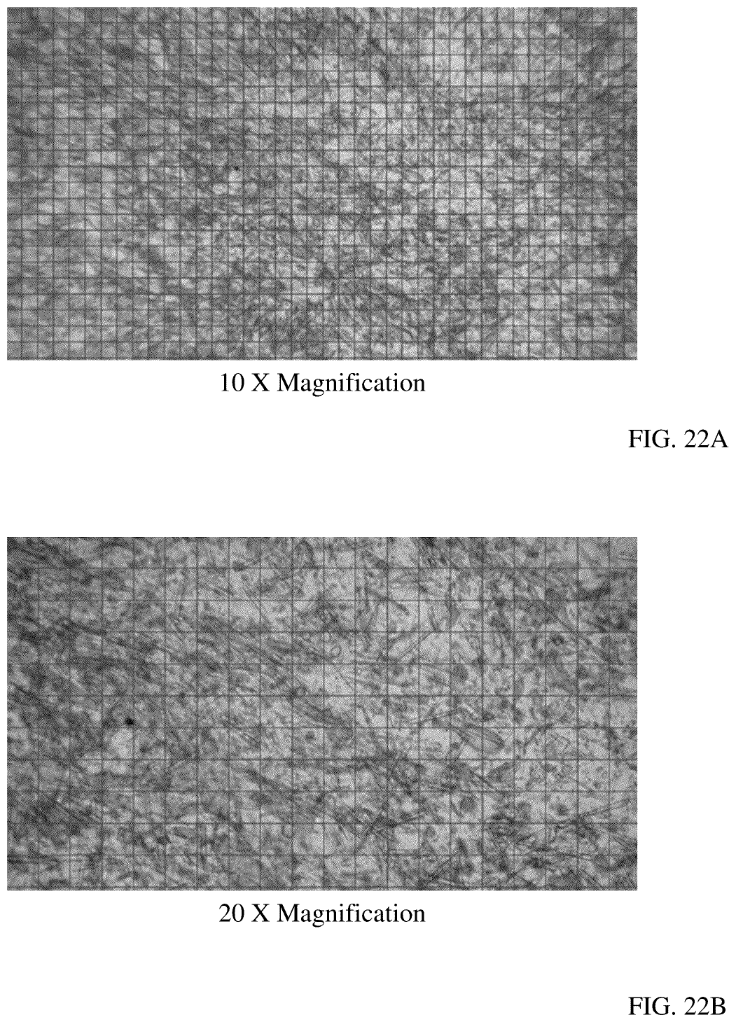

Sample 20 uses the base formulation but with 1.0% of the formulation consisting of carnauba wax and an equal reduction in soy oil. Images of the resulting material are shown under 10× magnification in FIG. 22A and under 20× magnification in FIG. 22B. The API crystals are long needle form, and the wax and lipids tend to coat the crystals. The product was a stable emulsion.

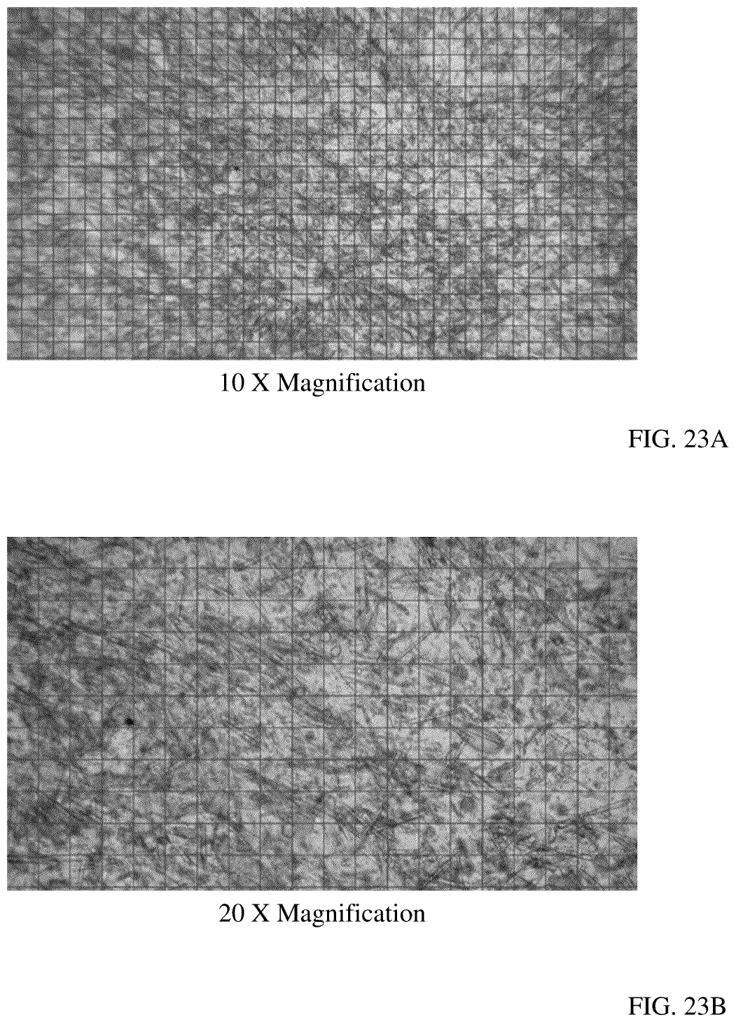

Sample 21 uses the base formulation but with 2.5% of the formulation consisting of carnauba wax and an equal reduction in soy oil. Images of the resulting material are shown under 10× magnification in FIG. 23A and under 20× magnification in FIG. 23B. The API crystals are long needle form. The wax and lipids coating on the crystals is thicker than seen in sample 20. The product was a stable emulsion.

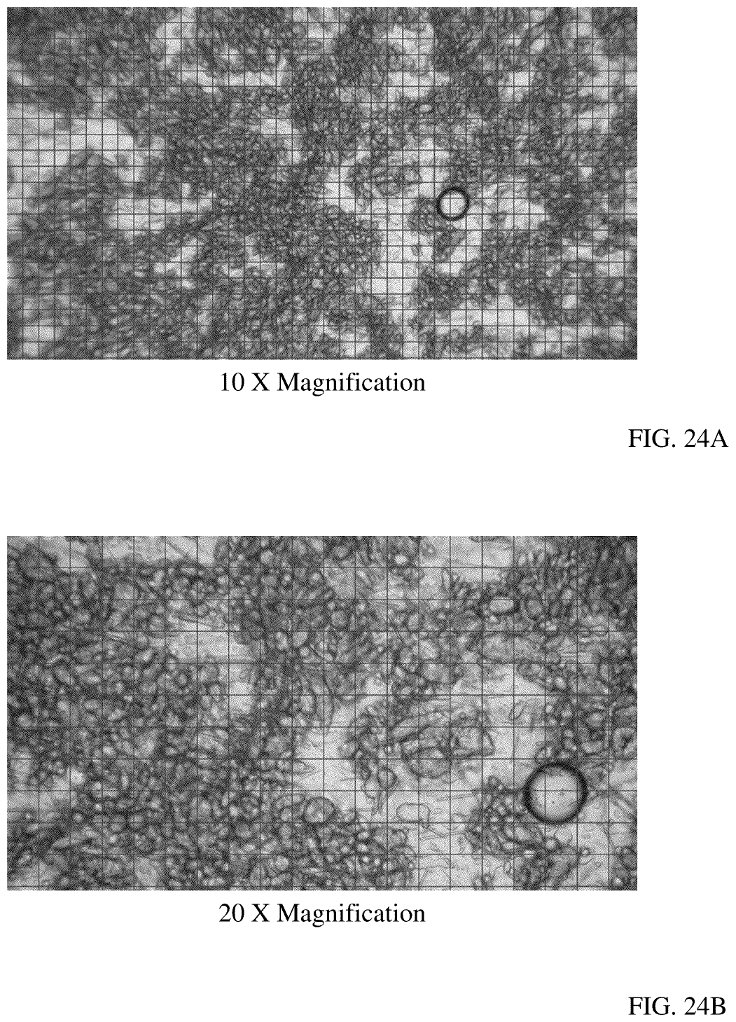

Sample 22 uses the base formulation but with 5.0% of the formulation consisting of carnauba wax and an equal reduction in soy oil. Images of the resulting material are shown under 10× magnification in FIG. 24A and under 20× magnification in FIG. 24B. The API crystals are long needle form. The wax and lipids have coated the crystals and have formed microparticles separate from the crystals. The product was a stable emulsion.

The carnauba wax appeared to coat much of the surface of the crystals. In higher concentrations, such as 5%, the carnauba wax also formed microparticles apart from the crystals. As would be understood, this tendency for the wax and lipids to coat the API crystals can lead to an increased barrier to diffusion of the API from the formulation into the subject. This increased barrier to diffusion can lead to a slower, more consistent release of the API into the subject. A slower more consistent release of the API allows for more of the formulation to be administered at a time, due to the lower rate of introduction to the subject.

### Example 8 Continuous Lipid Phase

In some implementations, and in this Example, it may be desirable to disperse the aqueous phase into a continuous lipid phase, rather than dispersing a lipid phase into a continuous aqueous phase, as in the other Examples. Having a continuous lipid phase may be beneficial when the API in use is hydrophilic, rather than hydrophobic. The formulation for sample 23 consisted of 60% lipid phase and 40% aqueous phase, by volume, with components of each phase kept in relative proportion, except where noted. The sodium hyaluronate in the formulation was 0.15% by weight, relative to the aqueous phase.

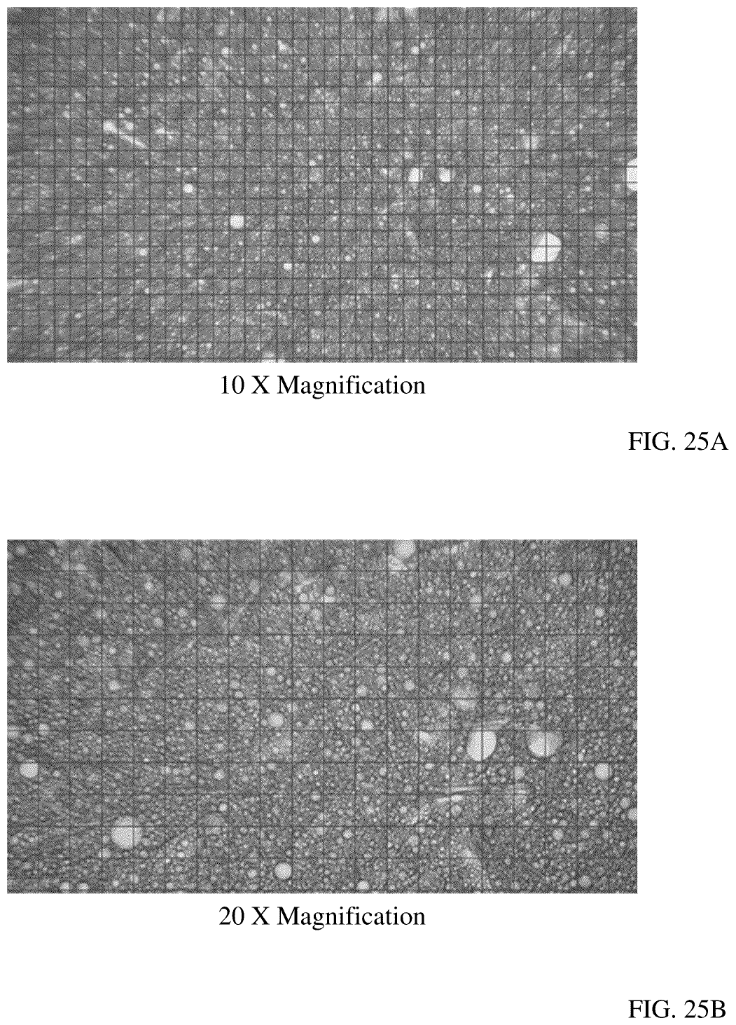

Images of the resulting material are shown under 10× magnification in FIG. 25A and under 20× magnification in FIG. 25B. The API crystals are long needle form. The aqueous phase droplets were varied in size.

### Example 9 Increased Mixing Energy

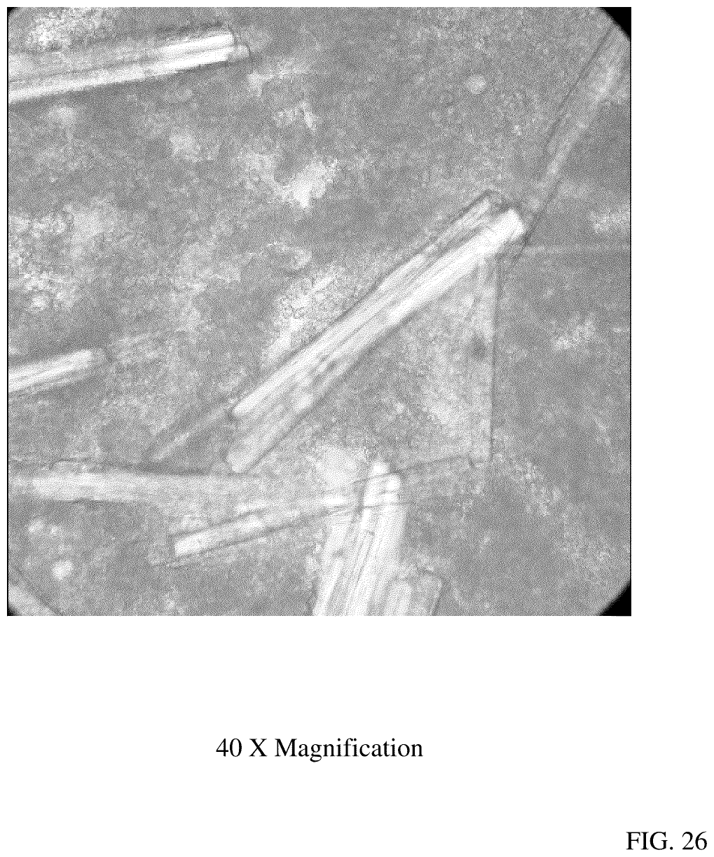

Sample 24 uses the base formulation but used more aggressive mixing energy. This was achieved by using a ¾ horsepower Silverson high-shear rotor-stator homogenizer at 6000 RPM. An image of the resulting material is shown under 40× magnification in FIG. 26. The oil droplets are smaller than those found in formulations made with standard mixing energy. The smaller oil droplets tend to coat the surface of the API crystals. The emulsion remained stable for more than 7 months.

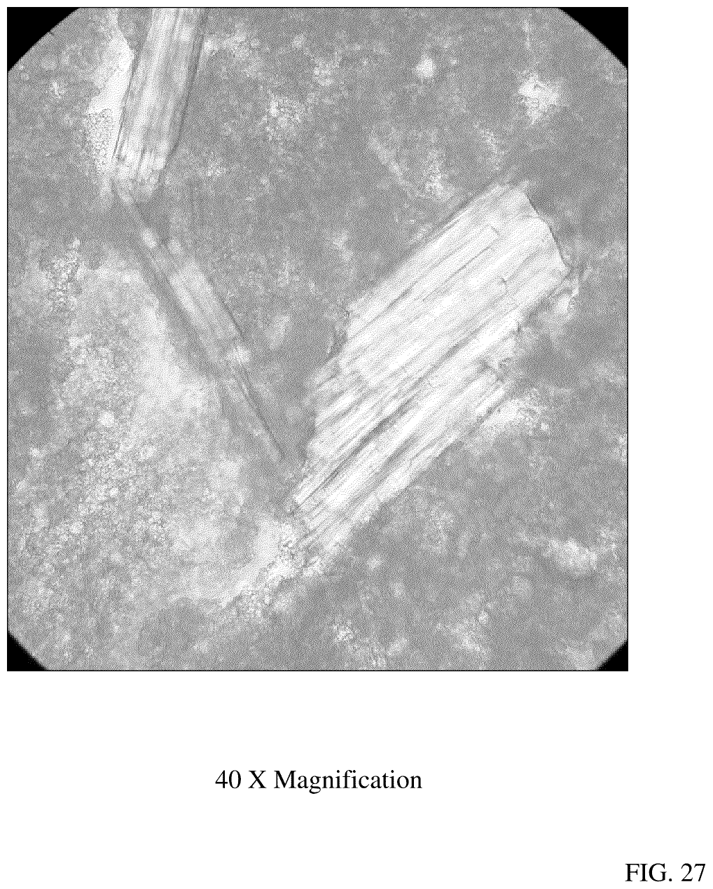

Sample 25 uses the base formulation but used the ¾ horsepower Silverson high-shear rotor-stator homogenizer at 12,000 RPM. An image of the resulting material is shown under 40× magnification in FIG. 27. API crystal aggregations formed as a result of the higher mixing energy. These aggregations remain stable and do not separate. The increased mixing energy also increased the size distribution of the API crystals and crystal agglomerates.

### Example 12—Increasing Stability with Increasing API Loading

In a comparison between a formulation containing no API (placebo) and a formulation containing API (active), it was observed that the placebo formulation had broken, while the active formulation had not. However, samples with higher API concentrations, in the form of ropivacaine, were less stable than those of lower concentrations. A sample with 29.34 mg/mL ropivacaine had greater stability than a sample with 38.51 mg/mL ropivacaine, which had greater stability than a sample with 47.69 mg/mL ropivacaine, which had greater stability than placebo. The 29.34 mg/mL ropivacaine sample is stable for at least 7 months.

### Example 13—Increasing Viscosity with API Loading

In a set of samples shown in Table 13, it can be seen that in certain embodiments, addition of an API, such as Ropivacaine, can lead to an increase in viscosity in the overall formulation. All of these samples in Table 13 use the Base Formulation, with the API loading varying as noted.

TABLE 13
Viscosity measurements.

| | | | | |
|---|---|---|---|---|
| API Concentration (mg/mL) | 0 | 29.37 | 38.51 | 47.68 |
| Viscosity (cP) | 124.0 | 249.6 | 199.6 | 215.4 |

Although the disclosure has been described with reference to preferred embodiments, persons skilled in the art will recognize that changes may be made in form and detail without departing from the spirit and scope of the disclosed apparatus, systems and methods.

What is claims is:

1. A composition for treating post-surgical pain in a subject in need thereof, comprising:
an emulsion comprising:
an aqueous carrier; and liquid lipid phase dispersed into droplets within the aqueous carrier, and a first anesthetic within the lipid phase.

2. The composition of claim 1, wherein the first anesthetic comprises a first plurality of anesthetic crystals.

3. The composition of claim 1, wherein the first anesthetic is dissolved with the lipid phase.

4. The composition of claim 2, further comprising a second plurality of anesthetic crystals within the aqueous carrier, but not the lipid phase and wherein the second plurality of anesthetic crystals dissolves and elutes from the emulsion at a faster rate than the first plurality anesthetic crystals.

5. The composition of claim 1, wherein the composition further comprises one or more additional anesthetics, different from the first anesthetic.

6. The composition of claim 1, wherein the lipid phase comprises a triglyceride.

7. The composition of claim 1, wherein the aqueous carrier further comprises a thickener and a polyol wherein the thickener is hyaluronic acid and wherein the polyol is glycerol and is present in an amount of from about 0.25 to about 2.5% (w/v) of the composition and wherein a thickener increases the viscosity of the composition.

8. The composition of claim 1, wherein the lipid phase further comprises a phospholipid present in an amount from about 0.1% to about 2.0% of the lipid phase.

9. The composition of claim 1, wherein the lipid phase is from about 10% to about 40% (w/v).

10. The composition of claim 1, wherein the stable emulsion further comprises lecithin, and wherein lecithin is present in amount of from 0.1-20% (w/v) of the emulsion.

11. The composition of claim 1, wherein the emulsion further comprises dextrose, and wherein dextrose is present in amount of from about 1-2% (w/v) of the emulsion.

12. The composition of claim 1, wherein the emulsion further comprises sorbitol, and wherein sorbitol is present in amount of from about 0.1-2% (w/v) of the emulsion.

13. The composition of claim 1, wherein the lipid phase comprises soybean oil in an amount from about 15% to 80% (v/v) and one or more medium chain triglycerides in amount from about 15% to about 20% (v/v).

14. The composition of claim 13, wherein the anesthetic is present in an amount from about 0.1 mg/g soybean oil to about 300 mg/g soybean oil.

15. The composition of claim 1, wherein the first anesthetic is selected lidocaine, prilocaine, mepivacaine, ropivacaine, etidocaine, levobupivacaine, bupivacaine, cocaine, procaine, 2-chloroprocaine, tetracaine, benzocaine, amethocaine, chlorocaine, butamben, dibucaine, and ester analogs of aconitine, dyclonine, ketamine, pramoxine, safrole, and salicyl alcohol.

16. The composition of claim 1, wherein the lipid phase comprises tributyrate and/or stearate.

17. The composition of claim 2, wherein the lipid phase further comprises an oil and/or wax that is solid at 25° C. in an amount of from about 5% to about 30% of the lipid phase (w/w) and wherein the oil and/or wax coats the anesthetic crystals.

18. The composition of claim 1, wherein the emulsion is stable for at least six months.

19. The composition of claim 1, wherein the anesthetic is ropivacaine and/or bupivacaine.

20. A composition for treating pain in a subject in need thereof, comprising:
an emulsion comprising:
a lipid carrier phase; and an aqueous phase dispersed into droplets within the lipid carrier phase, and a first anesthetic within the aqueous phase.

21. The composition of claim 20, wherein the first anesthetic comprises a first plurality of anesthetic crystals.

22. The composition of claim 21, further comprising a second plurality of anesthetic crystals present within the lipid carrier, but not the aqueous phase, and wherein the second plurality of anesthetic crystals dissolves and elutes from the emulsion at a faster rate than the first plurality anesthetic crystals.

23. The composition of claim 22, wherein the anesthetic is hydrophilic and is dissolved within the aqueous phase.

24. The composition of claim 20, wherein the composition further comprises one or more additional anesthetics, different from the first anesthetic.

25. A method of treating pain in a subject in need thereof comprising administering to the subject and effective amount of a composition comprising:
an emulsion comprising:
an aqueous carrier; and lipid phase dispersed into droplets within the aqueous carrier, and a first anesthetic within the lipid phase and wherein the anesthetic is eluted from the composition over a period of between about 4 and about 7 days.

26. The method of claim 25, wherein the anesthetic comprises a plurality of anesthetic crystals, and wherein the composition further comprises a second plurality of anesthetic crystals within the aqueous carrier, but not the lipid phase and wherein the second plurality of anesthetic crystals dissolves and elutes from the emulsion at a faster rate than the first plurality anesthetic crystals.

27. The method of claim 25, wherein the composition is delivered near a nerve or nerve bundle of a subject and wherein the nerve or nerve bundle innervates the surgical incision area of the subject.
